(19) United States
(12) Patent Application Publication
(10) Pub. No.: US 2024/0087122 A1
(43) Pub. Date: Mar. 14, 2024

(54) DETECTING TERTIARY LYMPHOID STRUCTURES IN DIGITAL PATHOLOGY IMAGES

(71) Applicant: Genentech, Inc., South San Francisco, CA (US)

(72) Inventors: Jennifer Margaret GILTNANE, Hillsborough, CA (US); Sertan KAYA, Fremont, CA (US); Cleopatra KOZLOWSKI, Woodside, CA (US); Geoffrey Fredrick SCHAU, Portland, OR (US)

(21) Appl. No.: 18/516,406

(22) Filed: Nov. 21, 2023

Related U.S. Application Data
(63) Continuation of application No. PCT/US2022/030978, filed on May 25, 2022.
(60) Provisional application No. 63/250,417, filed on Sep. 30, 2021.
(60) Provisional application No. 63/193,556, filed on May 26, 2021.

(51) Int. Cl. G06T 7/00; G06V 10/25; G06V 10/26; G06V 10/762; G06V 10/774; G06V 10/82; G06V 20/69; G16B 15/00

(52) U.S. Cl. CPC G06T 7/0012; G06V 10/25; G06V 10/26; G06V 10/762; G06V 10/774; G06V 10/82; G06V 20/695; G06V 20/698; G16B 15/00; G06T 2207/10056; G06T 2207/20081; G06T 2207/20084; G06T 2207/30024; G06T 2207/30096; G06V 2201/03

(57) ABSTRACT

In one embodiment, a method includes accessing a digital pathology image that depicts a tissue sample from a subject under a treatment, detecting tertiary lymphoid structures depicted within the digital pathology image of the tissue sample based on a machine-learning model, determining descriptive information associated with the detected tertiary lymphoid structures for the detected tertiary lymphoid structures, wherein the descriptive information comprises at least a maturation state associated with each of the detected tertiary lymphoid structures, and determining an outcome of the subject in response to the treatment based on the detected tertiary lymphoid structures and the descriptive information.

### CROSS-REFERENCE TO RELATED APPLICATIONS

This application claims the priority benefit of U.S. Provisional Patent Application Ser. No. 63/193,556, filed May 26, 2021, and of U.S. Provisional Patent Application Ser. No. 63/250,417, filed Sep. 30, 2021, the contents of each of which are incorporated herein by reference in their entirety.

### FIELD OF THE INVENTION

The present disclosure relates to a system and methods to detect tertiary lymphoid structures.

### INTRODUCTION

Tertiary lymphoid structures (TLS) are ectopic lymphoid formations that form within non-lymphoid tissue. Tertiary lymphoid structures (TLS) are frequently observed in organs targeted by autoimmune diseases. They share structural and functional characteristics with secondary lymphoid structures such as lymph nodes and can contain B-cell follicles and germinal centers surrounded by a T-cell region. TLS also present features such as segregated T and B cell zones, presence of follicular dendritic cell networks, high endothelial venules and specialized lymphoid fibroblasts, and display the mechanisms to support local adaptive immune responses toward locally displayed antigens. TLS have been described in several types of cancers and are usually associated with positive patient outcomes. However, TLS differ vastly in cellular composition and location within tissue types.

Tertiary lymphoid structures (TLS) develop in non-lymphoid tissues at sites of chronic inflammation, including within tumors. TLS exist under different maturation states in tumors, culminating in germinal center formation. The correlation between TLS presence and clinical benefit in patients with different types of cancer suggests that the presence of TLS could be a prognostic and predictive biomarker for treatment outcomes.

### SUMMARY OF PARTICULAR EMBODIMENTS

Disclosed herein are systems and methods to detect tertiary lymphoid structures (TLS).

The detection of tertiary lymphoid structures (TLS) in a tissue sample from a patient may oftentimes be prognostically beneficial for the patient as they may be more likely to respond to treatment, e.g., particularly in response to checkpoint inhibitor blockade immunotherapy. Conversely, the presence of TLS may confer a negative effect on patient outcome for some types of disease, e.g., in the indication of hepatocellular carcinoma. As a result, the presence of tertiary lymphoid structures in a patient tissue sample may serve as either a positive or negative biomarker depending on the indication.

Currently, tertiary lymphoid structures are manually identified in tissue samples by examination under a microscope or review of tissue sample slide images. Manual identification of tertiary lymphoid structures from slide images, e.g., by pathologists, can be time consuming as there may be only a small percentage of the tissue sample slides that comprise such structures. In addition, tertiary lymphoid structures may be hard to identify. For example, there can be a large variation in the appearance of actual tertiary lymphoid structures, and different pathologists often may not agree on whether or not a given slide image comprises a tertiary lymphoid structure. Furthermore, since tertiary lymphoid structures are a relatively new feature of possible interest, many pathologists have not been trained to identify tertiary lymphoid structures, or they may lack experience in identifying tertiary lymphoid structures. Consequently, current methods of identifying tertiary lymphoid structures are time-consuming and frequently unreliable.

If one can accurately identify the numbers and locations of tertiary lymphoid structures present in a tissue sample from a patient from slide images, it may be possible to predict the patient's response to treatment. To address this need, embodiments of the present disclosure include a digital pathology image processing system configured to perform automated detection of tertiary lymphoid structures in tissue slide images based on a TLS detection model that enables more effective and efficient TLS detection. In some instances, the ability to determine the maturation state of tertiary lymphoid structures may be particularly useful for determining a patient's response. Many patients may have the potential to form tertiary lymphoid structures that have not yet developed. Therefore, if one could, for example, deduce the maturation state of tertiary lymphoid structures, the TLS detection model may be able to more accurately determine exact locations of tertiary lymphoid structures, their sizes, and potentially other morphological factors such as maturation state. Such additional information, determined based on, e.g., a trained TLS detection model, may provide invaluable information for predicting patients' responses to cancer treatments.

In particular embodiments of the present disclosure, a digital pathology image processing system may access a digital pathology image that depicts a tissue sample from a subject under a treatment. The digital pathology image processing system may detect, based on a trained machine-learning model, one or more tertiary lymphoid structures within the tissue sample. In particular embodiments, the digital pathology image processing system may determine, for the one or more detected tertiary lymphoid structures, descriptive information associated with the detected tertiary lymphoid structures. The descriptive information may comprise at least a maturation state associated with each of the detected tertiary lymphoid structures. The digital pathology image processing system may further determine, based on the detected tertiary lymphoid structures and the descriptive information, a predicted outcome of the subject in response to the treatment.

Disclosed herein are methods comprising, by a digital pathology image processing system: accessing a digital pathology image that depicts a tissue sample from a subject under a treatment; detecting, based on a machine-learning model, one or more tertiary lymphoid structures depicted within the digital pathology image of the tissue sample; determining, for the one or more detected tertiary lymphoid structures, descriptive information associated with the detected tertiary lymphoid structures, wherein the descriptive information comprises at least a maturation state associated with each of the detected tertiary lymphoid structures; and determining, based on the detected tertiary lymphoid structures and the descriptive information, an outcome of the subject in response to the treatment.

In some embodiments, the method further comprises: identifying, for each of the one or more tertiary lymphoid structures, an area comprising the tertiary lymphoid structure; and providing instructions for outlining the area comprising the tertiary lymphoid structure in the digital pathology image, wherein the outlining comprises a polygonal shape. In some embodiments, the digital pathology image of the tissue sample depicts one or more structures, and the method further comprises: generating, for each of the one or more structures by the machine-learning model, a numeric representation, wherein detecting the one or more tertiary lymphoid structures comprises determining one or more of the structures as the one or more tertiary lymphoid structures based on the respective numeric representations associated with the one or more of the structures. In some embodiments, each of the one or more detected tertiary lymphoid structures is associated with a numeric representation generated by the machine-learning model, and the method further comprises: determining the maturation state associated with each of the detected tertiary lymphoid structures based on their respective numeric representations. In some embodiments, the digital pathology image of the tissue sample depicts one or more structures, and detecting the one or more tertiary lymphoid structures depicted within the digital pathology image of the tissue sample comprises: determining whether each of the one or more structures comprises a germinal center; and based on the determining of whether each of the one or more structures comprises a germinal center: if at least one of the one or more structures comprises a germinal center, determining the at least one structure as a tertiary lymphoid structure; else: analyzing an interaction between a presence of each of the one or more structures and one or more features associated with the structure; and determining whether each of the one or more structures is a tertiary lymphoid structure based on the analyzed interaction. In some embodiments, the digital pathology image of the tissue sample depicts one or more structures, and detecting the one or more tertiary lymphoid structures depicted within the digital pathology image of the tissue sample comprises: calculating, for each of the one or more structures identified by the machine-learning model, a confidence score based on a precision and recall for the machine-learning model, wherein the confidence score indicates a probability of the structure being a tertiary lymphoid structure; and determining, based on the one or more confidence scores, that one or more of the structures are tertiary lymphoid structures. In some embodiments, wherein the calculation used to determine confidence scores has been tuned to maximize the precision and recall of the machine-learning model when applied to a training set of annotated slide images. In some embodiments, the method further comprises: generating, for each of the one or more structures by the machine-learning model, a numeric representation; generating, one or more clusters of structures based on the numeric representation associated with each of the one or more structures; and updating the machine-learning model based on the one or more clusters associated with each of the one or more structures. In some embodiments, each of the determined one or more tertiary lymphoid structures is associated with a numeric representation, and the method further comprises: generating, one or more clusters of the detected tertiary lymphoid structures based on the numeric representation associated with each of the detected tertiary lymphoid structures; wherein determining the outcome of the subject in response to the treatment is further based on the one or more clusters. In some embodiments, the tissue sample is associated with one or more tumors, wherein the method further comprises: determining a type of the tissue sample; generating a tissue image mask for the tissue sample; identifying at least one tumor region within the tissue image mask; and determining a type of the at least one tumor region within the tissue image mask; wherein determining the outcome of the subject in response to the treatment is further based on the type of the tissue sample, the tissue image mask, and the type of the at least one tumor within the tissue image mask. In some embodiments, the tissue sample is associated with one or more tumors, and wherein the descriptive information further comprises one or more of: a number of the detected tertiary lymphoid structures; a number of detected tertiary lymphoid structures that are associated with image markers; a number of detected tertiary lymphoid structures outside a tumor region; a ratio between a number of detected tertiary lymphoid structures located inside the tumor region and a number of detected tertiary lymphoid structures located outside the tumor region; an average distance of the detected tertiary lymphoid structures to the tumor region or a boundary thereof; a size of each of the detected tertiary lymphoid structures; a percentage of a total tissue sample area that comprises the detected tertiary lymphoid structures; or an average distance between any given pair of detected tertiary lymphoid structures. In some embodiments, the machine-learning model is trained based on a plurality of training data, wherein each training data point comprises a slide image of a tissue sample and a corresponding annotation of tertiary lymphoid structures identified within that tissue sample. In some embodiments, the method further comprises training the machine-learning model, wherein the training comprises: applying one or more data augmentations to each slide image of a training data set, wherein the one or more data augmentations are based on one or more of brightness, hue, saturation, cropping, clipping, flipping, rotation, or a mean pixel density in a color channel. In some embodiments, the machine-learning model is based on one or more neural networks, and wherein the method further comprises training the machine-learning model, and wherein the training comprises: generating, by the one or more neural networks, one or more initial bounding boxes for each slide image, wherein each initial bounding box is associated with an initial aspect ratio and an initial size; adjusting, based on characteristics of tertiary lymphoid structures, the initial aspect ratio and the initial size associated with each of the initial bounding box; and training the machine-learning model based on slide image data using the adjusted bounding boxes. In some embodiments, the method further comprises training the machine-learning model, wherein the training comprises: identifying at least two tertiary lymphoid structures within at least one slide image of the training data; generating a cropped image from the at least one slide image by cropping the at least one slide image to make the at least two tertiary lymphoid structures centered; and training the machine-learning model based in part on the cropped image.

Also disclosed herein are one or more computer-readable non-transitory storage media embodying software that is operable when executed to: access a digital pathology image that depicts a tissue sample from a subject under a treatment; detect, based on a machine-learning model, one or more tertiary lymphoid structures depicted within the digital pathology image of the tissue sample; determine, for the one or more detected tertiary lymphoid structures, descriptive information associated with the detected tertiary lymphoid structures, wherein the descriptive information comprises at least a maturation state associated with each of the detected tertiary lymphoid structures; and determine, based on the detected tertiary lymphoid structures and the descriptive information, an outcome of the subject in response to the treatment. In some embodiments, the software is further operable when executed to: identify, for each of the one or more tertiary lymphoid structures, an area comprising the tertiary lymphoid structure; and provide instructions for outlining the area comprising the tertiary lymphoid structure in the digital pathology image, wherein the outlining comprises a polygonal shape. In some embodiments, the tissue sample comprises one or more structures, wherein the software is further operable when executed to: generate, for each of the one or more structures by the machine-learning model, a numeric representation, wherein detecting the one or more tertiary lymphoid structures comprises determining one or more of the structures as the one or more tertiary lymphoid structures based on the respective numeric representations associated with the one or more of the structures.

Disclosed herein are systems comprising: one or more processors; and a non-transitory memory coupled to the processors comprising instructions executable by the processors, the processors operable when executing the instructions to: access a digital pathology image that depicts a tissue sample from a subject under a treatment; detect, based on a machine-learning model, one or more tertiary lymphoid structures depicted within the digital pathology image of the tissue sample; determine, for the one or more detected tertiary lymphoid structures, descriptive information associated with the detected tertiary lymphoid structures, wherein the descriptive information comprises at least a maturation state associated with each of the detected tertiary lymphoid structures; and determine, based on the detected tertiary lymphoid structures and the descriptive information, an outcome of the subject in response to the treatment. In some embodiments, the processors are further operable when executing the instructions to: identify, for each of the one or more tertiary lymphoid structures, an area comprising the tertiary lymphoid structure; and provide instructions for outlining the area comprising the tertiary lymphoid structure in the digital pathology image, wherein the outlining comprises a polygonal shape.

### BRIEF DESCRIPTION OF THE DRAWINGS

FIG. 1 illustrates a network of interacting computer systems that can be used, as described herein according to some embodiments of the present disclosure.

FIG. 2A illustrates an exemplary method for detecting tertiary lymphoid structures (TLS).

FIG. 2B illustrates another exemplary method for detecting tertiary lymphoid structures (TLS).

FIG. 3 illustrates an example mature tertiary lymphoid structure with its annotation.

FIGS. 4A-4C illustrate additional examples of mature tertiary lymphoid structures.

FIG. 4A provides a first non-limiting example of a mature tertiary lymphoid structure. FIG. 4B provides a second non-limiting example of a mature tertiary lymphoid structure. FIG. 4C provides a third non-limiting example of a mature tertiary lymphoid structure.

FIG. 5 illustrates an example validation of the TLS detection model against five pathologists.

FIG. 6A illustrates an example agreement matrix between the TLS detection model and five pathologists.

FIG. 6B illustrates example viewer versus consensus statistical metrics.

FIG. 7 illustrates an example of a computing system.

### DESCRIPTION

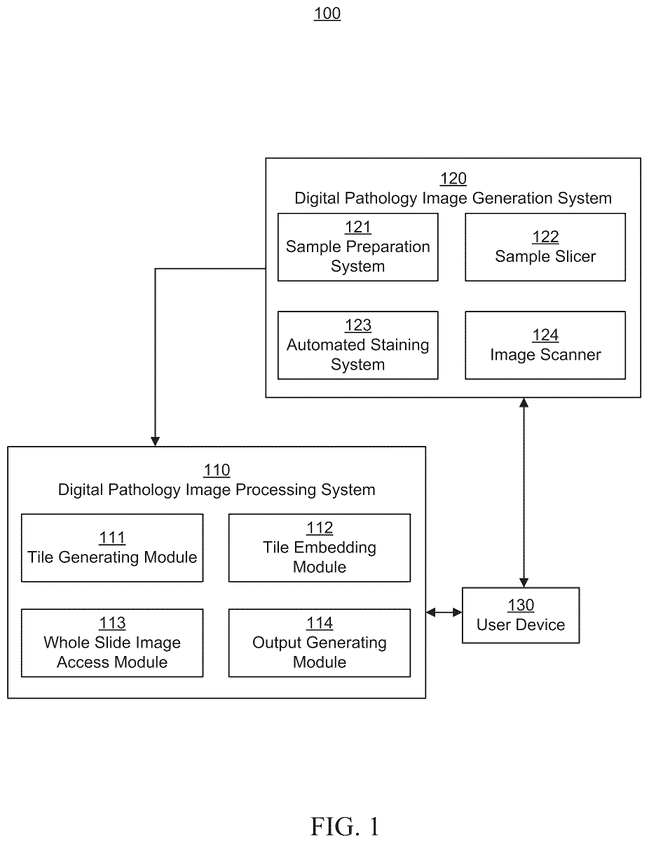

FIG. 1 illustrates a network 100 of interacting computer systems that can be used, as described herein according to some implementations of the systems of the present disclosure.

A digital pathology image generation system 120 can generate one or more whole slide images or other related digital pathology images, corresponding to a particular sample. For example, an image generated by digital pathology image generation system 120 can include a stained section of a biopsy sample. As another example, an image generated by digital pathology image generation system 120 can include a slide image of a liquid sample (e.g., a blood film). As another example, an image generated by digital pathology image generation system 120 can include a slide image depicting fluorescence in situ hybridization (FISH) after a fluorescent probe has been bound to a target DNA or RNA sequence.

Some types of samples (e.g., biopsies, solid samples and/or samples including tissue) can be processed by a sample preparation system 121 to fix and/or embed the sample. Sample preparation system 121 can facilitate infiltrating the sample with a fixating agent (e.g., liquid fixing agent, such as a formaldehyde solution) and/or embedding substance (e.g., a histological wax). For example, a sample fixation sub-system can fix a sample by exposing the sample to a fixating agent for at least a threshold amount of time (e.g., at least 3 hours, at least 6 hours, or at least 13 hours). A dehydration sub-system can dehydrate the sample (e.g., by exposing the fixed sample and/or a portion of the fixed sample to one or more ethanol solutions) and potentially clear the dehydrated sample using a clearing intermediate agent (e.g., that includes ethanol and a histological wax). A sample embedding sub-system can infiltrate the sample (e.g., one or more times for corresponding predefined time periods) with a heated (e.g., and thus liquid) histological wax. The histological wax can include a paraffin wax and potentially one or more resins (e.g., styrene or polyethylene). The sample and wax can then be cooled, and the wax-infiltrated sample can then be blocked out.

A sample slicer 122 can receive the fixed and embedded sample and can produce a set of sample sections. Sample slicer 122 can expose the fixed and embedded sample to cool or cold temperatures. Sample slicer 122 can then cut the chilled sample (or a trimmed version thereof) to produce a set of sections. Each section can have a thickness that is (for example) less than 100 μm, less than 50 μm, less than 10 μm or less than 5 μm. Each section can have a thickness that is (for example) greater than 0.1 μm, greater than 1 μm, greater than 2 μm or greater than 4 μm. The cutting of the chilled sample can be performed in a warm water bath (e.g., at a temperature of at least 30° C., at least 35° C. or at least 40° C.).

An automated staining system 123 can facilitate staining one or more of the sample sections by exposing each section to one or more staining agents. Each section can be exposed to a predefined volume of staining agent for a predefined period of time. In some instances, a single section is concurrently or sequentially exposed to multiple staining agents.

Each of one or more stained sections can be presented to an image scanner 124, which can capture a digital image of the section. Image scanner 124 can include a microscope and digital camera. The image scanner 124 can capture the digital image at multiple levels of magnification (e.g., 10×, 20×, 40×, etc.) using different objectives (e.g., a 10× objective, 20× objective, 40× objective, etc.). Manipulation of the image can be used to capture a selected portion of the sample at the desired range of magnifications. In some instances, image scanner 124 may be configured to perform bright-field imaging, dark-field imaging, phase contrast imaging, fluorescence imaging, or any combination thereof. Image scanner 124 can further capture annotations and/or morphometrics (e.g., quantitative characteristics of sample and morphology) identified by a human operator. In some instances, a section is returned to automated staining system 123 after one or more images are captured, such that the section can be washed, exposed to one or more other stains, and imaged again. When multiple stains are used, the stains can be selected to have different color profiles, such that a first region of an image corresponding to a first section portion that absorbed a large amount of a first stain can be distinguished from a second region of the image (or a different image) corresponding to a second section portion that absorbed a large amount of a second stain. Examples of suitable stains include, but are not limited to, hematoxylin and eosin (H & E) stain, periodic acid—Schiff (PAS) stain, prussian blue, alcian blue, oil red O, luxol fast blue—cresyl violet, toluidine blue, Van Kossa stain, Bodian stain, Giemsa stain, May-Grunwald Giemsa stain, benzidine stain, Feulgen stain, chromogenic- or fluorescently-labeled immunohistochemical stains, or any combination thereof.

It will be appreciated that one or more components of digital pathology image generation system 120 can, in some instances, operate in connection with human operators. For example, human operators can move the sample across various sub-systems (e.g., of sample preparation system 121, or of digital pathology image generation system 120) and/or initiate or terminate operation of one or more sub-systems, systems, or components of digital pathology image generation system 120. As another example, part or all of one or more components of digital pathology image generation system (e.g., one or more subsystems of the sample preparation system 121) can be partly or entirely replaced with actions of a human operator.

Further, it will be appreciated that, while various described and depicted functions and components of digital pathology image generation system 120 pertain to processing of a solid and/or biopsy sample, other embodiments can relate to processing of a liquid sample (e.g., a blood sample). For example, digital pathology image generation system 120 can receive a liquid-sample (e.g., blood or urine) slide, that includes a base slide, smeared liquid sample and cover. Image scanner 124 can then capture an image of the sample slide. Further embodiments of the digital pathology image generation system 120 can relate to capturing images of samples using advancing imaging techniques, such as immunofluorescence or FISH, described herein. For example, once a fluorescent probe has been introduced to a sample and allowed to bind to a target, e.g., a nucleic acid sequence, appropriate imaging can be used to capture images of the sample for further analysis.

A given sample can be associated with one or more users (e.g., one or more physicians, laboratory technicians and/or medical providers) during processing and imaging An associated user can include, by way of example and not of limitation, a person who ordered a test or biopsy that produced a sample being imaged, a person with permission to receive results of a test or biopsy, or a person who conducted analysis of the test or biopsy sample, among others. For example, a user can correspond to a physician, a pathologist, a clinician, or a subject. A user can use one or more user devices 130 to submit one or more requests (e.g., that identify a subject) that a sample be processed by digital pathology image generation system 120 and/or that a resulting image be processed by a digital pathology image processing system 110.

Digital pathology image generation system 120 can transmit an image produced by image scanner 124 back to user device 130. User device 130 may then communicate with the digital pathology image processing system 110 to initiate automated processing of the image. In some instances, digital pathology image generation system 120 provides an image produced by image scanner 124 to the digital pathology image processing system 110 directly, e.g. at the direction of the user of a user device 130. Although not illustrated, other intermediary devices (e.g., data stores of a server connected to the digital pathology image generation system 120 or digital pathology image processing system 110) can also be used. Additionally, for the sake of simplicity only one digital pathology image processing system 110, image generating system 120, and user device 130 is illustrated in the network 100. This disclosure anticipates the use of one or more of each type of system and component thereof without necessarily deviating from the teachings of this disclosure.

The network 100 and associated systems shown in FIG. 1 can be used in a variety of contexts where scanning and evaluation of digital pathology images, such as whole slide images, are an essential component of the work. As an example, the network 100 can be associated with a clinical environment, where a user is evaluating the sample for possible diagnostic purposes. The user can review the image using the user device 130 prior to providing the image to the digital pathology image processing system 110. The user can provide additional information to the digital pathology image processing system 110 that can be used to guide or direct the analysis of the image by the digital pathology image processing system 110. For example, the user can provide a prospective diagnosis or preliminary assessment of features within the scan. The user can also provide additional context, such as the type of tissue being reviewed. As another example, the network 100 can be associated with a laboratory environment where tissues are being examined, for example, to determine the efficacy or potential side effects of a drug. In this context, it can be commonplace for multiple types of tissues to be submitted for review to determine the effects on the whole body of said drug. This can present a particular challenge to human scan reviewers, who may need to determine the various contexts of the images, which can be highly dependent on the type of tissue being imaged. These contexts can optionally be provided to the digital pathology image processing system 110.

Digital pathology image processing system 110 can process digital pathology images, including whole slide images, to classify the digital pathology images and generate annotations for the digital pathology images and related output. As an example, the digital pathology image processing system 110 can process whole slide images of tissue samples or tiles (e.g., selected regions) of the whole slide images of tissue samples generated by the digital pathology image processing system 110, to detect tertiary lymphoid structures and determine the maturation state of these tertiary lymphoid structures. The digital pathology image processing system 110 may generate masks of TLS and non-TLS. As an example and not by way of limitation, the digital pathology image processing system 110 may generate, for each of the detected tertiary lymphoid structures, a polygon surrounding the tertiary lymphoid structure. The digital pathology image processing system 110 may crop the queried whole slide image into a plurality of image tiles. A tile generating module 111 can define a set of tiles for each digital pathology image. To define the set of tiles, the tile generating module 111 can segment the digital pathology image into the set of tiles. As embodied herein, the tiles can be non-overlapping (e.g., each tile includes pixels of the image not included in any other tile) or overlapping (e.g., each tile includes some portion of pixels of the image that are included in at least one other tile). Features such as whether or not tiles overlap, in addition to the size of each tile and the step-size (e.g., the image distance or number of pixels between a tile and a subsequent tile) used to shift a window used for segmenting the image can increase or decrease the data set for analysis, with more tiles (e.g., through overlapping or smaller tiles) increasing the potential resolution of eventual output and visualizations. In some instances, tile generating module 111 defines a set of tiles for an image where each tile is of a predefined size and/or an offset between tiles is predefined. Continuing with the example of detecting tertiary lymphoid structures, each slide image may be cropped into image tiles having a width and height of a certain number of pixels. Furthermore, in some instances, the tile generating module 111 can create multiple sets of tiles of varying size, varying degree of overlap, varying step size, etc., for each image. As an example, the width and height of an image tile in terms of number of pixels may be dynamically determined (i.e., not fixed) based on factors such as the evaluation task, the queried image itself, or any suitable factor. In some embodiments, overlapping tiles may be analyzed. Uniform segmentation of the whole slide image, without tile overlapping, can be a preferable solution to balance tile processing requirements and avoid influencing the embedding generation and weighting value generation discussed herein. A tile size or tile offset can be determined, for example, by calculating one or more model performance metrics (e.g., a precision, recall, accuracy, and/or error associated with detection of TLS in a training set of annotated slide images) for each size/offset and by selecting a tile size and/or offset associated with one or more performance metrics above a corresponding predetermined threshold and/or associated with one or more performance metric categories(s) (e.g., high precision, high recall, high accuracy, and/or low error).

The tile generating module 111 may further define a tile size depending on the type of abnormality being detected. For example, the tile generating module 111 can be configured with awareness of the type(s) of tissue abnormalities that the digital pathology image processing system 110 will be searching for, and can customize the tile size according to the specific tissue abnormality to improve detection. For example, the tile generating module 111 can determine that, when searching for, e.g., inflammation or necrosis in lung tissue, the tile size should be reduced, while when searching for, e.g., Kupffer cells in liver tissues, the tile size should be increased. In some instances, the tile size may be increased or decreased to adjust scanning rate. In some instances, tile generating module 111 defines a set of tiles where a number of tiles in the set, size of the tiles of the set, resolution of the tiles for the set, or other related properties, for each image is defined and held constant for each of one or more images.

As embodied herein, the tile generating module 111 can further define the set of tiles for each digital pathology image along one or more color channels or color combinations. As an example, digital pathology images received by digital pathology image processing system 110 can include large-format multi-color channel images having pixel color values for each pixel of the image specified for one of several color channels. Example color specifications or color spaces that can be used include the RGB, CMYK, HSL, HSV, or HSB color specifications. The set of tiles can be defined based on segmenting the color channels and/or generating a brightness map or greyscale equivalent of each tile. For example, for each segment of an image, the tile generating module 111 can provide a red tile, blue tile, green tile, and/or brightness tile, or the equivalent for the color specification used. As explained herein, segmenting the digital pathology images based on segments of the image and/or color values of the segments can improve the accuracy and recognition rates of the neural networks used to generate embeddings for the tiles and image and to produce classifications of the image. Additionally, the digital pathology image processing system 110, e.g., using tile generating module 111, can convert between color specifications and/or prepare copies of the tiles using multiple color specifications. Color specification conversions can be selected based on a desired type of image augmentation (e.g., accentuating or boosting particular color channels, saturation levels, brightness levels, etc.). Color specification conversions can also be selected to improve compatibility between digital pathology image generation systems 120 and the digital pathology image processing system 110. For example, a particular image scanning component can provide output in the HSL color specification and the machine-learning models used in the digital pathology image processing system 110, as described herein, can be trained using RGB images. Converting the tiles to the compatible color specification can ensure the tiles can still be analyzed. Additionally, the digital pathology image processing system can up-sample or down-sample images that are provided in particular color depth (e.g., 8-bit, 1-bit, etc.) to be usable by the digital pathology image processing system. Furthermore, the digital pathology image processing system 110 can cause tiles to be converted according to the type of image that has been captured (e.g., fluorescent images may include greater detail on color intensity or a wider range of colors).

As described herein, a tile embedding module 112 can generate an embedding for each tile in a corresponding feature embedding space. The embedding can be represented by the digital pathology image processing system 110 as a feature vector for the tile. The tile embedding module 112 can use a neural network (e.g., a convolutional neural network) to generate a feature vector that represents each tile of the image. In particular embodiments, the tile embedding neural network can be based on the ResNet image network trained on a dataset based on natural (e.g., non-medical) images, such as the ImageNet dataset. By using a non-specialized tile embedding network, the tile embedding module 112 can leverage known advances in efficiently processing images to generate embeddings. Furthermore, using a natural image dataset allows the embedding neural network to learn to discern differences between tile segments on a holistic level.

In other embodiments, the tile embedding network used by the tile embedding module 112 can be an embedding network customized to handle large numbers of tiles derived from large format images, such as digital pathology whole slide images. Additionally, the tile embedding network used by the tile embedding module 112 can be trained using a custom dataset. For example, the tile embedding network can be trained using a variety of samples of whole slide images or even trained using samples relevant to the subject matter for which the embedding network will be generating embeddings (e.g., scans of particular tissue types). Training the tile embedding network using specialized or customized sets of images can allow the tile embedding network to identify finer differences between tiles, which can result in more detailed and accurate determination of distances between tiles in the feature embedding space (at the cost of additional time to acquire the images used for training, and the computational and economic cost of training multiple tile generating networks for use by the tile embedding module 112). In some instances, the tile embedding module 112 can select from a library of trained tile embedding networks based on the type of images being processed by the digital pathology image processing system 110.

As described herein, tile embeddings can be generated from a deep learning neural network using visual features of the tiles. Tile embeddings can be further generated from contextual information associated with the tiles or from the content shown in the tile. For example, a tile embedding can include one or more features that indicate and/or correspond to a size of depicted objects (e.g., sizes of depicted cells or aberrations) and/or density of depicted objects (e.g., a density of depicted cells or aberrations). Size and density can be measured absolutely (e.g., height or width expressed in pixels, or converted from pixels to nanometers) or relative to other tiles from the same digital pathology image, from a class of digital pathology images (e.g., produced using similar techniques or by a single digital pathology image generation system or scanner), or from a related family of digital pathology images. Furthermore, tiles can be classified prior to the tile embedding module 112 generating embeddings for the tiles such that the tile embedding module 112 considers the classification when preparing the embeddings.

For consistency, the tile embedding module 112 may produce embeddings of a predefined size (e.g., vectors of 512 elements, vectors of 2048 bytes, etc.). The tile embedding module 112 can also produce embeddings of various and arbitrary sizes. The tile embedding module 112 can adjust the sizes of the embeddings based on user direction or can be selected, for example, based on computation efficiency, embedding accuracy, or other parameters. In particular embodiments, the embedding size can be based on the limitations or specifications of the deep learning neural network that generated the embeddings. Larger embedding sizes can be used to increase the amount of information captured in the embedding and improve the quality and accuracy of results, while smaller embedding sizes can be used to improve computational efficiency.

The digital pathology image processing system 110 can perform different inferences by applying one or more machine-learning models (e.g., supervised or unsupervised machine learning models) to the embeddings, i.e., inputting the embeddings to one or more machine-learning models. As an example, the digital pathology image processing system 110 can detect, based on a machine-learning model trained to detect tertiary lymphoid structures, a tertiary lymphoid structure. In some embodiments, it may not be necessary to crop the image into image tiles, generate embeddings for these tiles, and then perform inferences based on such embeddings. Instead, the digital pathology image processing system 110 can directly apply the machine-learning model to the embedding of a whole slide image to make inferences provided that the system has sufficient GPU memory. In some instances, the output of the machine-learning model may be resized and formatted in the shape of the input image.

A whole slide image access module 113 can manage requests to access whole slide images from other modules of the digital pathology image processing system 110 and the user device 130. For example, the whole slide image access module 113 may receive requests to identify a whole slide image based on a particular tile, an identifier for the tile, or an identifier for the whole slide image. The whole slide image access module 113 can perform tasks of confirming that the whole slide image is available to the user requesting the image, identifying the appropriate databases from which to retrieve the requested whole slide image, and retrieving any additional metadata that may be of interest to the requesting user or module. Additionally, the whole slide image access module 113 can handle efficiently streaming the appropriate data to the requesting device. As described herein, whole slide images may be provided to user devices in segments, based on the likelihood that a user will wish to see only a relevant portion of the whole slide image. The whole slide image access module 113 can determine which regions of the whole slide image to provide and determine how to provide them. Furthermore, the whole slide image access module 113 can be empowered within the digital pathology image processing system 110 to ensure that no individual component locks up or otherwise misuses a database or whole slide image to the detriment of other components or users.

An output generating module 114 of the digital pathology image processing system 110 can generate output corresponding to result tile and result whole slide image datasets based on a user request. As described herein, the output can include a variety of visualizations, interactive graphics, and reports based upon the type of request and the type of data that is available. As an example and not by way of limitation, the output may comprise a numeric readout that can be used to reference against patient outcome/response. For example, the numeric readout may comprise the number of tertiary lymphoid structures detected in a slide image, the number of tertiary lymphoid structures with image markers (e.g., labels to identify individual TLS within the image), the number of tertiary lymphoid structures outside a tumor region of the sample, the ratio of tertiary lymphoid structures inside and outside the tumor region, the average distance of tertiary lymphoid structures to a tumor region or tumor region boundary, the size of tertiary lymphoid structures, the percentage of the total image area comprising tertiary lymphoid structures, the average distance between any given pair of tertiary lymphoid structures, etc. In many embodiments, the output will be provided to the user device 130 for display, but in certain embodiments the output can be accessed directly from the digital pathology image processing system 110. The output will be based on existence of and access to the appropriate data, so the output generating module will be empowered to access necessarily metadata and anonymized patient information as needed. As with the other modules of the digital pathology image processing system 110, the output generating module 114 can be updated and improved in a modular fashion, so that new output features can be provided to users without requiring significant downtime.

The general techniques described herein can be integrated into a variety of tools and use cases. For example, as described, a user (e.g., pathology or clinician) can access a user device 130 that is in communication with the digital pathology image processing system 110 and provide a query image for analysis. The digital pathology image processing system 110, or the connection to the digital pathology image processing system can be provided as a standalone software tool or package that searches for corresponding matches, identifies similar features, and generates appropriate output for the user upon request. As a standalone tool or plug-in that can be purchased or licensed on a streamlined basis, the tool can be used to augment the capabilities of a research or clinical lab. Additionally, the tool can be integrated into the services made available to the customers of digital pathology image generation system manufacturers or to the customers of digital pathology image generation service providers. For example, the tool can be provided as part of a unified workflow, where a user who conducts or requests a whole slide image to be created automatically receives an report of noteworthy features within the image and/or similar whole slide images that have been previously indexed. Therefore, in addition to improving whole slide image analysis, the techniques can be integrated into existing systems to provide additional features not previously considered or possible.

Moreover, the digital pathology image processing system 110 can be trained and customized for use in particular settings. For example, the digital pathology image processing system 110 can be specifically trained for use in providing insights relating to specific types of tissue (e.g., lung, heart, blood, liver, etc.). As another example, the digital pathology image processing system 110 can be trained to assist with safety assessment, for example in determining levels or degrees of toxicity associated with drugs or other potential therapeutic treatments. Once trained for use in a specific subject matter or use case, the digital pathology image processing system 110 is not necessarily limited to that use case. Training may be performed in a particular context, e.g., toxicity assessment, due to a relatively larger set of at least partially-labeled or annotated images.

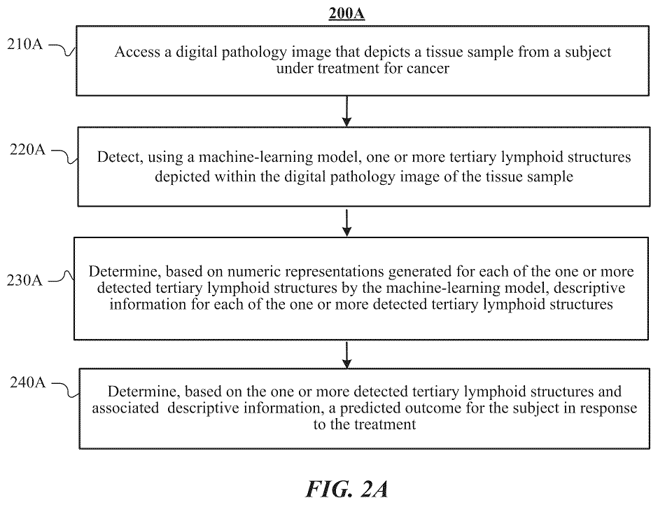

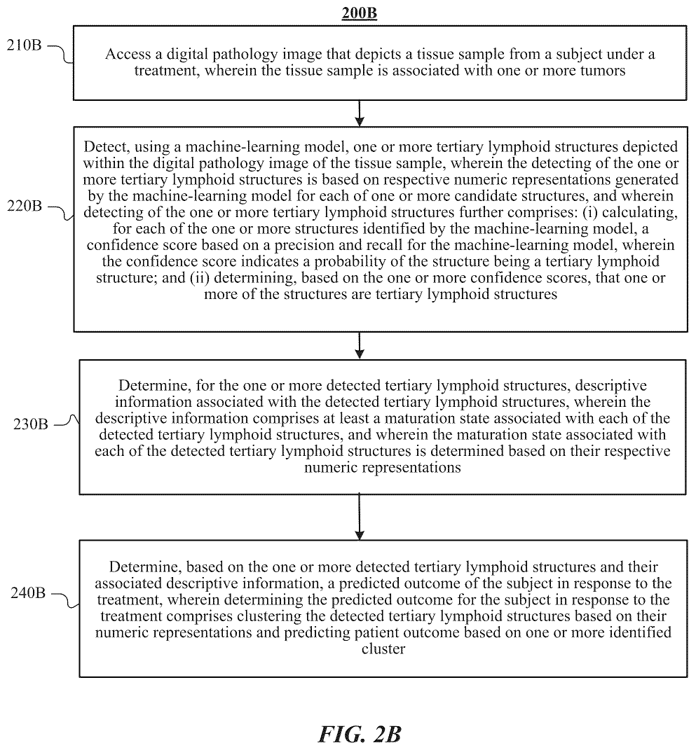

FIG. 2A illustrates an exemplary method 200A for detecting tertiary lymphoid structures and predicting treatment outcomes. The method may begin at step 210A, where the digital pathology image processing system 110 may access a digital pathology image that depicts, e.g., a tissue sample from a subject (e.g., human or animal) under a treatment. In alternative embodiments, the tissue sample may be taken from, e.g., pre-treatment biopsies, healthy subjects (e.g., human or animal), etc. The digital pathology image of the tissue sample may depict one or more structures, e.g., suspected or candidate TLS. As an example and not by way of limitation, the tissue sample may be derived from a subject suspected of having or diagnosed with a cancer. The digital pathology image may be a scanned, stained (e.g., hematoxylin and eosin stained) whole slide image of a tissue sample comprising tumorous cells (e.g., lung adenocarcinoma cells).

At step 220A, the digital pathology image processing system 110 may detect, using a trained machine-learning model, one or more tertiary lymphoid structures depicted within the digital pathology image of the tissue sample. In particular embodiments, detecting the one or more tertiary lymphoid structures depicted within the digital pathology image of the tissue sample may be based on respective numeric representations associated with the one or more candidate structures. In some instances, the respective numeric representations associated with the one or more candidate structures may be generated by the machine-learning model as described elsewhere herein.

At step 230A, the digital pathology image processing system 110 may determine descriptive information associated with the one or more detected tertiary lymphoid structures based on, for example, the numeric representations generated by the machine learning model. The descriptive information may comprise, for example, a list of features associated with each tertiary lymphoid structure (e.g., size, area, distance to tumor, etc.) and a list of features associated with the whole slide image (e.g., average TLS size, average distance, total area, etc.). In particular embodiments, the descriptive information may comprise at least a determination of maturation state associated with each of the detected tertiary lymphoid structures.

At step 240A, the digital pathology image processing system 110 may determine, based on the detected tertiary lymphoid structures and/or the associated descriptive information, a predicted outcome of the subject in response to the treatment. In some instances, the prediction of an outcome for the subject in response to treatment based on the detected tertiary lymphoid structures and/or the associated descriptive information may be performed using one or more additional trained machine-learning models, i.e., machine-learning model(s) that are different from the trained model used for detection of TLS.

FIG. 2B illustrates another exemplary method 200B for detecting tertiary lymphoid structures. The method may begin at step 210A, where the digital pathology image processing system 110 may access a digital pathology image that depicts, e.g., a tissue sample from a subject (e.g., human or animal) under a treatment. In alternative embodiments, the tissue sample may be taken from, e.g., pre-treatment biopsies, health subjects (e.g., human or animal), eic. The digital pathology image of the tissue sample may depict one or more structures, e.g., suspected or candidate TLS. As an example and not by way of limitation, the tissue sample may be associated with one or more tumors in a subject suspected of having or diagnosed with a cancer. The digital pathology image may be a scanned, stained (e.g., hematoxylin and eosin stained) whole slide image of a tissue sample comprising tumorous cells (e.g., lung adenocarcinoma cells).

At step 220B, the digital pathology image processing system 110 may detect, using a machine-learning model-based analysis of the digital pathology image, one or more tertiary lymphoid structures depicted within the image of the tissue sample. In particular embodiments, detecting the one or more tertiary lymphoid structures depicted within the digital pathology image of the tissue sample may be based on respective numeric representations associated with the one or more of the structures. The respective numeric representations may be generated by the machine-learning model. In particular embodiments, detecting the tertiary lymphoid structures based on numeric representations may include detecting tumor lesion structures to measure distances between tertiary lymphoid structures and tumors (e.g., between tertiary lymphoid structures and tumor centroids, or between tertiary lymphoid structures and tumor boundaries), detecting lymph node structures to filter out TLS detections made by the TLS detection model within lymph nodes (i.e., a potential source of error for the TLS detection model), In some embodiments, detecting the one or more tertiary lymphoid structures depicted within the digital pathology image of the tissue sample may alternatively comprise, or may further comprise, calculating, for each of the one or more structures detected by the machine-learning model, a confidence score indicating a probability of the structure being a tertiary lymphoid structure, determining a precision and a recall based on the one or more confidence scores of the one or more structures, and determining, based on the precision and the recall, that one or more of the structures are tertiary lymphoid structures.

At step 230B, the digital pathology image processing system 110 may determine, for the one or more detected tertiary lymphoid structures, descriptive information associated with the detected tertiary lymphoid structures. The descriptive information may comprise a list of features associated with each tertiary lymphoid structure (e.g., size, area, distance to tumor, distance to tumor centroid, distance to a neared tumor boundary, etc.) and a list of features associated with the whole slide image (e.g., average TLS size, average distance, total area, total tumor area, etc.). In particular embodiments, the descriptive information may comprise at least a maturation state associated with each of the detected tertiary lymphoid structures. Mature TLS may be identified, for example, based on the presence of a germinal center, the presence of a distinct T cell zone, and/or the presence of a network of CD23-positive dendritic cells. The maturation state associated with each of the detected tertiary lymphoid structures may be determined based on their respective numeric representations. In particular embodiments, the descriptive information may further comprise one or more of a number of the detected tertiary lymphoid structures, a number of detected tertiary lymphoid structures that are associated with image markers, a number of detected tertiary lymphoid structures outside the tumors, a ratio between detected tertiary lymphoid structures inside the tumors and detected tertiary lymphoid structures outside the tumors, an average distance of the detected tertiary lymphoid structures to the tumors, a size of each of the detected tertiary lymphoid structures, a percentage of an area comprising the detected tertiary lymphoid structures over an entire area of the tissue sample, or an average distance between any given pair of detected tertiary lymphoid structures. Besides the aforementioned human-interpretable features, the embodiments disclosed herein may quantify additional TLS-based features apart from human-interpretable features such as size of the tertiary lymphoid structure, area comprising the tertiary lymphoid structures, distances between tertiary lymphoid structures, etc. As an example and not by way of limitation, the TLS detection model may use an instance segmentation approach, which may learn an abstract representation of each TLS instance. Such abstract representations may be not easily interpreted by humans. These abstract representations may capture features such as texture, tone, brightness, contrast, or gradient, as well as complex geometric features such as structural regularity/irregularity. Similar abstract representations may be also enabled through the use of semi-supervised learning methods, such as autoencoders, which may be designed to ingest images of tertiary lymphoid structures for the same purpose of learning meaningful representations of the tertiary lymphoid structures.

At step 240B, the digital pathology image processing system 110 may determine, based on the detected tertiary lymphoid structures and the descriptive information, a predicted outcome of the subject in response to the treatment. In particular embodiments, determining the predicted outcome of the subject in response to the treatment may comprise generating one or more clusters of the detected tertiary lymphoid structures based on the numeric representation associated with each of the detected tertiary lymphoid structures. Accordingly, determining the predicted outcome of the subject in response to the treatment may be further based on the one or more clusters of tertiary lymphoid structures identified in the image. In particular embodiments, the digital pathology image processing system 110 may further determine a type of the tissue sample, generate a tissue image mask for the tissue sample (e.g., a binary mask that identifies tumor regions within the image), identify at least one tumor region within the tissue image mask, and determine a type of the at least one tumor region within the tissue image mask. Accordingly, determining the predicted outcome of the subject in response to the treatment may be further based on the type of the tissue sample, the tissue image mask, and the type of the at least one tumor (or tumor region) identified within the tissue image mask. Particular embodiments may repeat one or more steps of the method of FIG. 2B, where appropriate. Although this disclosure describes and illustrates particular steps of the method of FIG. 2B as occurring in a particular order, this disclosure contemplates any suitable steps of the method of FIG. 2B occurring in any suitable order. Moreover, although this disclosure describes and illustrates an example method for detecting tertiary lymphoid structures, including the particular steps of the method of FIG. 2B, this disclosure contemplates any suitable method for detecting tertiary lymphoid structures, including any suitable steps, which may include all, some, or none of the steps of the method of FIG. 2B, where appropriate. Furthermore, although this disclosure describes and illustrates particular components, devices, or systems carrying out particular steps of the method of FIG. 2B, this disclosure contemplates any suitable combination of any suitable components, devices, or systems carrying out any suitable steps of the method of FIG. 2B.

The detection of tertiary lymphoid structures (TLS) may be prognostically beneficial for a patient as they may be more likely to respond to treatment if tertiary lymphoid structures are present in their tissues. If one can accurately identify the numbers and locations of these tertiary lymphoid structures from slide images, it may be possible to predict a patient's response to treatment. However, as noted above, manual identification of tertiary lymphoid structures from slide images, e.g., by pathologists, may be time consuming as there may be only a small percentage of tissue sample slides that comprise such structures. In addition, tertiary lymphoid structures may be hard to identify. For example, there can be a large variation in the appearance of actual tertiary lymphoid structure, and different pathologists often may not agree on whether or not a given slide image comprises a tertiary lymphoid structure. As a result, a digital pathology image processing system 110 may use an automated approach based on a trained TLS detection model to detect tertiary lymphoid structures from slide images more effectively and efficiently. In addition, the maturation state of tertiary lymphoid structures may be particularly useful for determining a patient's response. Many patients may have the potential to form tertiary lymphoid structures that have not yet developed. Therefore, if one could, for example, induce the maturation state of tertiary lymphoid structures, the TLS detection model may be able to more accurately determine exact locations of tertiary lymphoid structures, their sizes, and potentially other morphological factors such as maturation state. Such additional information determined based on, e.g., a trained TLS detection model may provide invaluable information for predicting patients' responses to treatment.

In particular embodiments, a digital pathology image processing system 110 may access a digital pathology image that depicts a tissue sample from a subject under a treatment. The digital pathology image processing system 110 may detect, based on a machine-learning model, one or more tertiary lymphoid structures depicted within the digital pathology image of the tissue sample. In particular embodiments, the digital pathology image processing system 110 may determine, for the one or more detected tertiary lymphoid structures, descriptive information associated with the detected tertiary lymphoid structures. The descriptive information may comprise at least a maturation state associated with each of the detected tertiary lymphoid structures. The digital pathology image processing system 110 may further determine, based on the detected tertiary lymphoid structures and the descriptive information, an outcome of the subject in response to the treatment.

In particular embodiments, the digital pathology image processing system 110 may determine, based on the output of the TLS detection model, a numeric readout/descriptive information that can be used to reference against patient outcome/response. As an example and not by way of limitation, the numeric readout/descriptive information may comprise one or more of a number of the detected tertiary lymphoid structures, a number of detected tertiary lymphoid structures that are associated with image markers, a number of detected tertiary lymphoid structures located outside of tumor tissue, a ratio between the number of detected tertiary lymphoid structures located inside tumors and the number of detected tertiary lymphoid structures located outside of the tumors, an average distance of the detected tertiary lymphoid structures to the tumors (e.g., an average distance of the detected TLS to a tumor centroid, or to a nearest tumor boundary), a size of each of the detected tertiary lymphoid structures, a percentage of an area comprising the detected tertiary lymphoid structures over an entire area of the tissue sample, or an average distance between any given pair of detected tertiary lymphoid structures. The digital pathology image processing system 110 may further translate the numeric readout/descriptive information into a quantitative biomarker, which may be referred as human interpretable features (HIFs), e.g., TLS-specific HIFs. Me HIFs may be associated with other clinical data and assay read-outs, comprising one or more of mutation status. RNA-sequence, or patient demographics. As an example and not by way of limitation, a set of human interpretable features may comprise a table with rows indicating features and columns indicating patient outcomes/responses. The digital pathology image processing system 110 may then reference against patient outcomes/responses.

In particular embodiments, the digital pathology image processing system 110 may use another machine-learning model to predict patient outcome/response based on the TLS-specific HIFS. As an example and not by way of limitation, the number of tertiary lymphoid structures detected inside the tumor region divided by the total number of tertiary lymphoid structures that have been identified may be an important indicator for predicting patient outcome/response for one patient but not for another patient. There may be a plurality of TLS-specific HIFS, and a variety of combinations thereof, some of which may serve as important prognostic biomarkers while others do not. In some instances, the machine-learning model used to predict patient outcome/response may be a non-linear model to effectively account for the relationship between a plurality of TLS-specific HIFS, and combinations thereof, and predicted patient outcome/response.

In particular embodiments, the digital pathology image processing system 110 may deploy the TLS detection model in parallel with other models, e.g., a tumor-lesion detection model, to generate a comprehensive readout of a slide image. The digital pathology image processing system 110 may determine a type of the tissue sample, generate a tissue image mask for the tissue sample, identify at least one tumor or tumor region within the tissue image mask, and determine a type of the at least one tumor or tumor region within the tissue image mask. As an example and not by way of limitation, the TLS detection model may generate masks of TLS and non-TLS whereas the tumor-lesion detection model may fit a convex hull or polygon around the exact perimeter of a tumor lesion. Accordingly, the digital readout may comprise a tissue image mask, the type of the tissue, the type of tumor or tumor region identified within that tissue image mask, the tertiary lymphoid structures detected within that tissue image mask, tertiary lymphoid structures clustering data, etc. In particular embodiments, all of these masks (e.g., masks for TLS and masks for tissues) and data derived therefrom may be translated into predictive/prognostic biomarkers for medical analysis. As an example and not by way of limitation, determining the outcome of the subject in response to the treatment may be further based on the type of the tissue sample, the tissue image mask, and the type of the at least one tumor identified within the tissue image mask.

In particular embodiments, each structure within a slide image may have a structure representation generated during the processing by the TLS detection model. As an example and not by way of limitation, the structure representation may be numerical, which may be generated by, e.g., a neural network of the TLS detection model. Given that the digital pathology image of the tissue sample depicts one or more structures, the digital pathology image processing system 110 may generate, for each of the one or more structures by the machine-learning model, a numeric representation. Accordingly, detecting the one or more tertiary lymphoid structures may comprise determining that one or more of the structures are tertiary lymphoid structures based on the respective numeric representations associated with the one or more of the structures. The digital pathology image processing system 110 may further determine, e.g., the maturation state associated with each of the detected tertiary lymphoid structures based on their respective numeric representations.

In particular embodiments, the digital pathology image processing system 110 may extract these numerical representations for post-processing. Tertiary lymphoid structures that share similar structures may share similar numerical representations, for which a clustering approach may be used to group tertiary lymphoid structures that have a similar appearance. As a result, the digital pathology image processing system 110 may cluster the structures based on the numerical representations of these structures. The digital pathology image processing system 110 may further leverage different TLS clusters, or data derived therefrom, to determine therapeutic or strategic benefits from an oncology biomarker development perspective. In other words, determining the outcome of the subject in response to the treatment may be further based on the one or more TLS clusters identified in the tissue sample from the subject. As an example and not by way of limitation, cluster A may comprise tertiary lymphoid structures having a large germinal center, cluster B may comprise tertiary lymphoid structures having a small germinal center, and cluster C may comprise tertiary lymphoid structures being oblong. The digital pathology image processing system 110 may analyze each cluster and compare them against each other. For example, the digital pathology image processing system 110 may identify cluster A comprising three tertiary lymphoid structures and cluster B comprising two tertiary lymphoid structures. The digital pathology image processing may pair such information with, e.g., an unsupervised clustering algorithm to further discriminate these tertiary lymphoid structures.

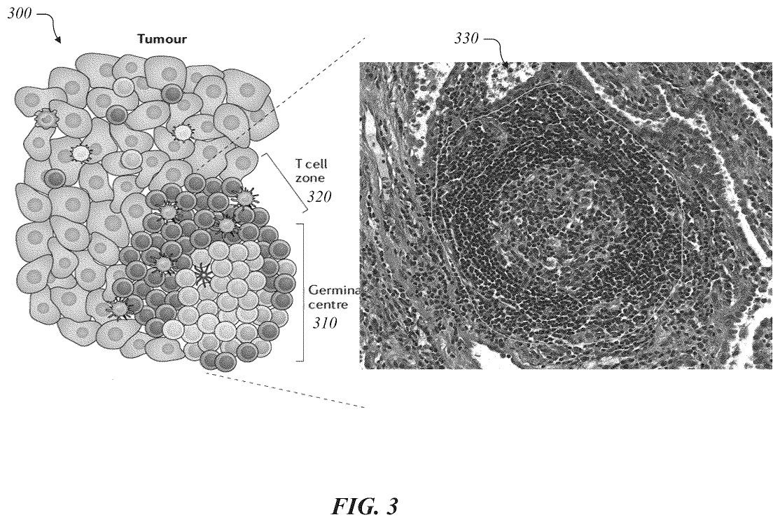

FIG. 3 illustrates an example of a mature tertiary lymphoid structure with its annotation. Mature tertiary lymphoid structures 300 may have a three-dimensional structure with a germinal center 310 in the middle and different types of cells, e.g., the T cell zone 320 surrounded by lymphocytes. Therefore, detecting the one or more tertiary lymphoid structures depicted within the digital pathology image of the tissue sample may begin with determining whether the one or more structures comprises a germinal center. If at least one of the one or more structures is determined to comprise a germinal center, the digital pathology image processing system 110 may identify the at least one structure as a tertiary lymphoid structure. If the structure comprises a group of lymphocytes, it may indicate a mature tertiary lymphoid structure 300 or an immature tertiary lymphoid structure that may eventually develop into a mature tertiary lymphoid structure 300. The ability to objectively distinguish between these scenarios may be used as part of the morphological characterization used to predict a patient's response.

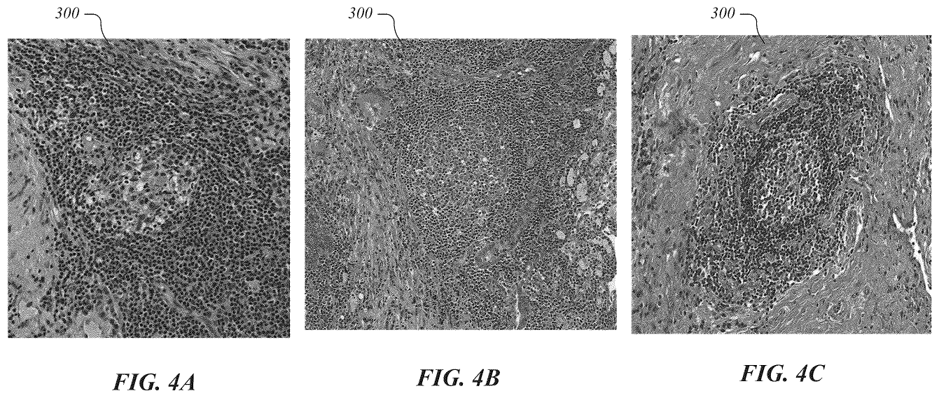

FIGS. 4A-4C illustrate additional example mature tertiary lymphoid structures 300. In FIGS. 4A-4C, the slide images of the tertiary lymphoid structures 300 were stained by H&E (hematoxylin and eosin). In particular embodiments, the digital pathology image processing system 110 may utilize the numeric representations of structures generated by the TLS model to determine whether they are mature tertiary lymphoid structures 300, immature tertiary lymphoid structures, or not tertiary lymphoid structures, from a relatively unbiased perspective. One may tell the maturation state of a tertiary lymphoid structure from the morphology of the tertiary lymphoid structure and the morphology may be encoded in the numeric representation of the structure. As a result, by analyzing the numeric representation, the digital pathology image processing may determine the maturation state of detected TLS. In particular embodiments, the numeric representation may be an abstract representation of a structure identified in the slide image, which the digital pathology image processing system 110 may transform to, for example, a one-dimensional vector. In some instances, the digital pathology image processing system 110 may project the representation into, e.g., a spectral space, where it may be compared to a spectrum indicating maturation state of tertiary lymphoid structures. As an example and not by way of limitation, one end of the spectrum may indicate a mature tertiary lymphoid structure 300, so if the projection of the numeric representation is near this end of the spectrum, the corresponding structure may be a mature tertiary lymphoid structure 300. As another example and not by way of limitation, the mid-range of the spectrum may indicate an immature tertiary lymphoid structure, so if the projection of the representation is near the mid-range of the spectrum, the corresponding structure may be an immature tertiary structure. As yet another example and not by way of limitation, the other end of the spectrum may indicate a lymphoid aggregate rather than a tertiary lymphoid structure, so if the projection of the representation is near this end of the spectrum, the corresponding structure may be not a tertiary lymphoid structure.

If there is no indication of a germinal center in an identified structure, one may not be able to determine whether it is a mature tertiary lymphoid structure 300 or whether it is a tertiary lymphoid structure at all (e.g., lymphoid aggregates may look alike). To be able to detect mature tertiary lymphoid structures 300 when there is no discernible germinal center 310, the digital pathology image processing system 110 may analyze, for example, how the presence of an identified structure correlates with other features, e.g., morphological features, identifiable using digital pathology. In other words, based on determining whether one or more identified structures comprises a germinal center: if none of the one or more structures comprises a germinal center, the digital pathology image processing system 110 may analyze a correlation or interaction between a presence of each of the one or more structures and one or more features, e.g., morphological features, associated with the structure and determine whether each of the one or more structures is a tertiary lymphoid structure based on the analyzed correlation/interaction. For example, in some instances the digital pathology image processing system 110 may use a tumor detection algorithm and a phenotyping algorithm to analyze lymphocyte infiltration in tumors, e.g., T-cell and/or B-cell infiltration. The relative number, distribution, and role of the T-cells versus the B-cells in the structure may be helpful for detecting and identifying mature tertiary lymphoid structures.

In particular embodiments, the digital pathology image processing system 110 may use an alternative or additional approach to help determine the maturation state of a tertiary lymphoid structure. The digital pathology image processing system 110 may take a first H&E (hematoxylin and eosin) slide image together with a neighboring slide image (e.g., a slide image that depicts another one or more structures located near the structure depicted in the first H&E slide), determine TLS-specific markers (e.g., morphological features or numeric representations thereof) for both slides, and analyze the first H&E slide based on these TLS-specific markers, which may result in a more accurate way to determine if a structure is a tertiary lymphoid structure and what it's maturation state may be.

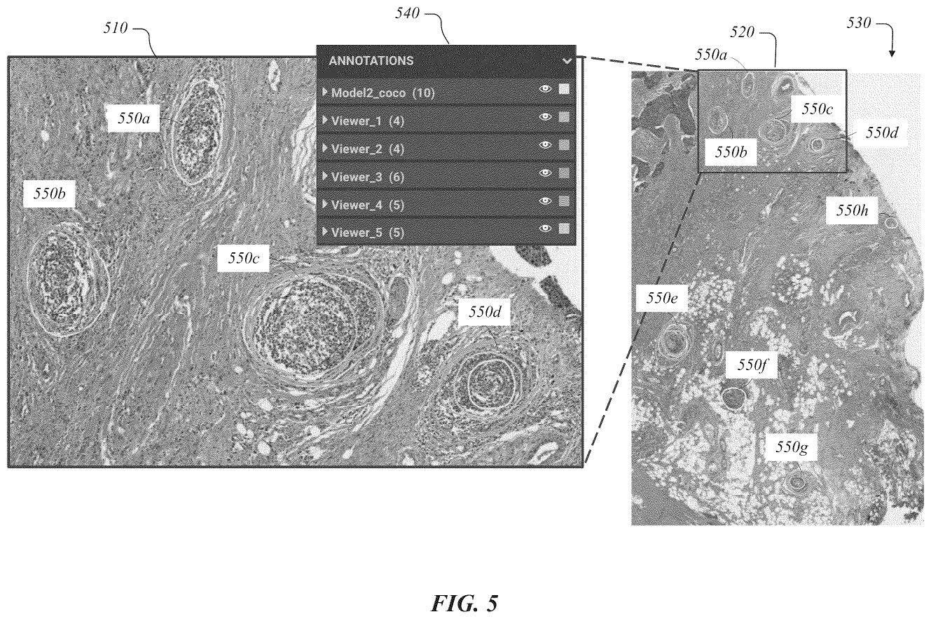

FIG. 5 illustrates an example of a validation study that compared the predictions of the TLS detection model against five pathologists. The left part 510 of FIG. 5 depicts a zoomed-in view of a part 520 of the whole slide image 530. The statistics panel 540 comprises a listing of the annotations generated by the TLS detection model and the five pathologists, and indicates that the TLS detection model was able to detect and annotate 10 tertiary lymphoid structures, the first and second pathologists (viewer_1 and viewer_2) were able to find and annotate 4 tertiary lymphoid structures 300, the third pathologist (viewer_3) was able to find and annotate 6 tertiary lymphoid structures 300, and the fourth and fifth pathologists (viewer_4 and viewer_5) were able to find and annotate 5 tertiary lymphoid structures. As an example and not by way of limitation, the TLS detection model was able to annotate tertiary lymphoid structures 550a-550h (the remaining two tertiary lymphoid structures are not shown in FIG. 5) while the pathologists could only find some of them. The validation study may indicate that the TLS detection model is more effective in detecting TLS than human effort. The validation study may also verify the discrepancy among pathologists regarding what may be called an actual tertiary lymphoid structure.

In particular embodiments, the sensitivity/specificity of the TLS detection model may alter depending on certain user-controlled parameters. For example, in one low-specificity high-sensitivity mode of operation, the TLS detection model may generate false positives while capturing most of the true tertiary lymphoid structures. In another high-specificity low-sensitivity mode of operation, the TLS detection model may have detected only true tertiary lymphoid structures but may have missed some, resulting in false negatives. In yet another mode of operation, the sensitivity/specificity trade-off may be optimized using some other parameter, such as maximizing the harmonic mean of sensitivity and specificity (e.g., the F1 score) to balance the expected error. Once a threshold is chosen by any of these above (or other) methods, agreement between the predictions of the TLS model and those made by a group of pathologists may be calculated to determine consensus.

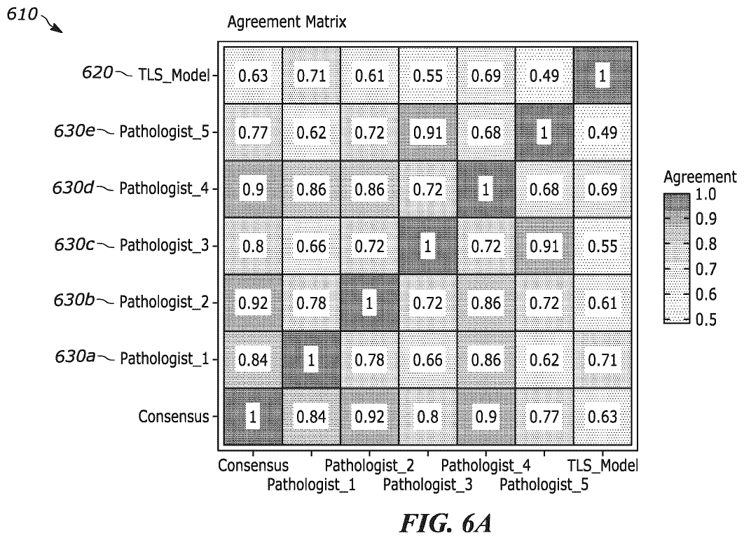

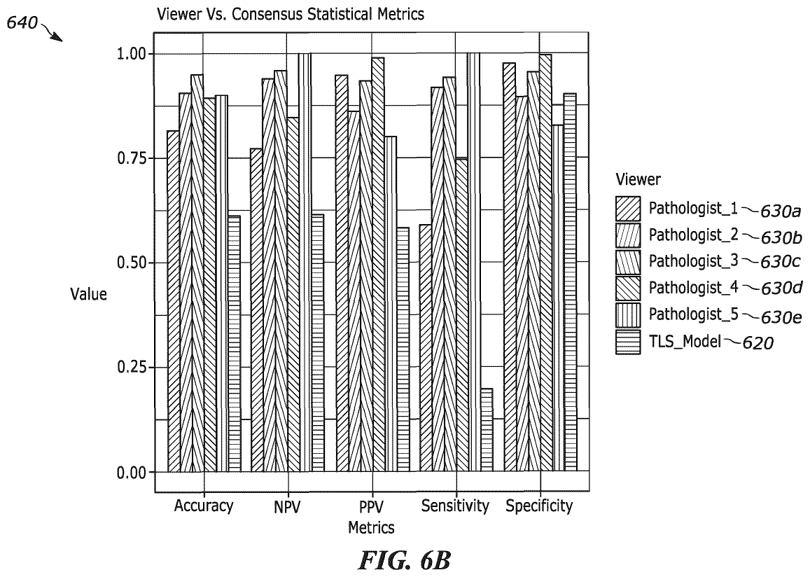

FIGS. 6A-6B illustrate examples of consensus data for comparisons of the performance of the TLS detection model and pathologists. FIG. 6A illustrates an example agreement matrix 610 between the results obtained by the TLS detection model (TLS_Model) 620 and five pathologists 630a-630e. FIG. 6B illustrates exemplary viewer versus consensus statistical metrics 640. FIGS. 6A-6B illustrate various ways to compare pathologists' ground truth to the pathologist consensus. As may be seen, the consensus between pathologists is not perfect, and the TLS detection model achieves similar error rates as a typical pathologist may make with respect to the consensus of, in this case, 5 pathologists. From FIGS. 6A-6B, one may see if individual pathologists are more similar to their consensus (assumed to be the most “correct”) than the TLS detection model is to the consensus. FIG. 6B shows results of a statistical analysis that is based on the agreement data depicted in FIG. 6A. The plot provided in FIG. 6B illustrates the relative performance (in terms of accuracy, negative predictive value (NPV), positive predictive value (PPV), clinical sensitivity, and clinical specificity) of the TLS detection model compared to the pathologists. This analysis is important because we use pathologist annotations as “ground truth”. In this example, the data indicate that the accuracy of the TLS detection model did not exceed the individual pathologists' accuracy, and provides an indication of how far the current version of the TLS detection model is from the maximum attainable accuracy.

In particular embodiments, the digital pathology image processing system 110 may be used to generate one or more training data sets, each comprising a plurality of training data points used to train the TLS detection model. Each training data point may comprise, for example, a slide image of a tissue sample and a corresponding annotation by a pathologist of tertiary lymphoid structures identified within that tissue sample. As an example and not by way of limitation, the training data may comprise stained (e.g. with H&E stain) slide images associated with tertiary lymphoid structures. The training data may also comprise annotations of tertiary lymphoid structures identified within the stained slide images by pathologists. The right-hand part of FIG. 3 illustrates an example annotation 330 of the tertiary lymphoid structure 300 on the left. The annotation 330 may comprise a polygon, i.e., a line drawn around the tertiary lymphoid structure 300. When annotating the slide images with these TLS polygons, the pathologists may follow a strict protocol that defines how the pathologists should annotate. The protocol may comprise a description of what magnification a pathologist should use to start their review of the slide image. The protocol may additionally comprise a description of what magnification they may use to zoom in and to confirm that an identified structure is a tertiary lymphoid structure. As an example and not by way of limitation, the pathologist may start at 2× magnification. The pathologist may then zoom in to approximately 10× magnification to confirm what they see. At that magnification, specific elements of a tertiary lymphoid structure may be evident, e.g., at a camera resolution of about 0.9 microns per pixel. In particular embodiments, based on an expected field of view and expected pixel resolution, the digital pathology image processing system 110 may infer the size of an image window that needs to be extracted from the original image in order for pathologists to be able to identify tertiary lymphoid structures and the spatial resolution of the zoomed image that is required for them to confirmation their identification of a tertiary lymphoid structure. The digital pathology image processing system 110 may then transform these annotations and image requirements into masks. In particular embodiments, a mask may be a binary overlay, which highlights what a pathologist considers to be a tertiary lymphoid structure in a given slide image used for training the TLS detection model.

In particular embodiments, the digital pathology image processing system 110 may provide a tool for pathologists to easily view the slide images and annotate them. There may be additionally a backend in the application program interface (API) that allows the digital pathology image processing system 110 to easily access the annotated data and other relevant data from the tool used by the pathologists.

The digital pathology image processing system 110 may then pipe the accessed annotated data (i.e., the training data) into a machine-learning architecture for training the TLS detection model. In some instances, the TLS detection model may be based on an artificial neural network or deep learning algorithm. As an example and not by way of limitation, the architecture may be based on a convolutional neural network (CNN) or a mask-based (or region-based) convolutional neural network (RCNN) configured for instance segmentation (i.e., the task of detecting and delineating structures of interest (e.g., TLS) in the tissue sample image according to the embodiments disclosed herein). Training the TLS detection model on a sufficient amount of data comprising slide images and their associated annotations and masks may enable the TLS detection model to identify tertiary lymphoid structures in non-annotated slide images. As an example and not by way of limitation, the output of the TLS detection model may comprise an annotated image with a polygon (e.g., a fine line) drawn around the detected tertiary lymphoid structure in the annotated slide image.

In particular embodiments, the digital pathology image processing system 110 may apply one or more data augmentations to each training slide image before inputting them into the machine-learning architecture for training. For example, the digital pathology image processing system 110 may take a slide image and the corresponding mask and modulate them to enhance the variability of the training data set that the TLS detection model is trained on. In particular embodiments, the one or more data augmentations may be based on one or more of brightness, hue, or saturation. In some instances, the data augmentations may be based on a distribution of pixel intensity in a given color channel or based on the mean pixel density in a given color channel. For example, the digital pathology image processing system 110 may compute the mean pixel intensity of the red, green and/or blue channels and scale individual pixel intensities accordingly. In particular embodiments, the one or more data augmentations may be also comprise image cropping, clipping, flips, and rotations, etc., which the aim of diversifying the training slide images used to train the TLS detection model. In particular embodiments, the data augmentation may comprise transforming one or more slide images to conform to bounds established based on a plurality of slide images. For example, for a training set comprising 100 slide images, one might estimate the distribution of brightness for the majority (e.g., the 99 percentile) of the slide images (or of the total population of image pixels in the training set) and shift the image brightness for the remaining slide images (or outlying image pixels) according to those bounds.

Biologically, tertiary lymphoid structures may be spherical or non-spherical. In particular embodiments, the TLS detection model may be based on one or more neural networks. In some instances, the neural networks (e.g., mask RCNN) may randomly generate bounding boxes in an image. Each bounding box may then be analyzed by the neural networks individually to evaluate if there is a target (e.g., a tertiary lymphoid structure) located in the bounding box. In particular embodiments, when training the TLS detection model based on such neural networks, the digital pathology image processing system 110 may generate, by the one or more neural networks, one or more initial bounding boxes for each slide image. Each initial bounding box may be associated with an initial aspect ratio (e.g., an aspect ratio of about 0.25, 0.5, 0.75, 1, 1.25, 1.5, 2, 3, or 4; or any value within this range) and an initial size (e.g., a maximum dimension of about 20 μm, 40 μm, 60 μm, 80 μm, 100 μm, 200 μm, 400 μm, 600 μm, or 800 μm; or any value within this range). The digital pathology image processing system 110 may then adjust, based on characteristics of tertiary lymphoid structures, the initial aspect ratio and/or the initial size associated with each of the initial bounding boxes. As an example and not by way of limitation, the digital pathology image processing system 110 may tighten up the bounding boxes significantly, e.g., by adjusting the aspect ratio for each bounding box to fall between 0.5 and 2, because the tertiary lymphoid structures are spherical. In particular embodiments, each tile within a slide image may be associated with a physical width, e.g., 1, 2, 3, or 4 millimeters. Therefore, the digital pathology image processing system 110 may also adjust the initial aspect ratio and initial size of the bounding boxes accordingly. The aspect ratio and size of the bounding boxes may then be adjusted such that they fit within a biological range of an annotated TLS area, e.g., an aspect ratio of about 0.5, 0.75, 1, 1.25, 1.5, 2 (or any value within this range) and a maximum dimensions of about 0.5 μm, 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, or 10 μm. The digital pathology image processing system 110 may further train the machine-learning model based on the adjusted bounding boxes.

There may be multiple (e.g., three or four) distinct tertiary lymphoid structures centered around a given tertiary lymphoid structure. In particular embodiments, the digital pathology image processing system 110 may perform data augmentation for adjacent tertiary lymphoid structures. Suppose there are multiple tertiary lymphoid structures located next to each other. The digital pathology image processing system 110 may identify at least two tertiary lymphoid structures within at least one slide image of the training data. The digital pathology image processing system 110 may then generate a cropped image from the at least one slide image by cropping the at least one slide image to make the at least two tertiary lymphoid structures centered in the cropped image. The digital pathology image processing system 110 may further train the machine-learning model based in part on the cropped image. However, if there are any other neighbor structures, the digital pathology image processing system 110 may appropriately mask at all these instances of neighbor structures in the image.

For overlapping tiles in the slide images, the digital pathology image processing system 110 may iteratively process each of the overlapping tiles to avoid producing artifacts. In particular embodiments, the digital pathology image processing system 110 may take all overlapping tiles and collapse them (i.e., produce a single flattened image tile). As a result, the digital pathology image processing system 110 may effectively generate another mask of the overlapped tiles as a backup. Each of the overlapping or non-overlapping full slide images may be processed by the digital pathology image processing system 110 using the TLS detection model. The TLS detection model may first perform an instance/structure segmentation pass to identify tertiary lymphoid structures. In particular embodiments, the digital pathology image processing system 110 may then perform instance/structure representation extraction. For each TLS instance identified by the TLS detection model, it may draw a boundary (e.g., a polygon) around that tertiary lymphoid structure. More specifically, the digital pathology image processing system 110 may identify, for each of the one or more tertiary lymphoid structures, an area comprising the tertiary lymphoid structure. The digital pathology image processing system 110 may further provide instructions for outlining the area comprising the tertiary lymphoid structure in the digital pathology image. As an example and not by way of limitation, the outline may comprise a polygonal shape.

In particular embodiments, the digital pathology image processing system 110 may perform retraining or fine-tuning of the TLS detection model based on the detection results for a training data set. The detection results of the model may comprise false positives and false negatives, as measured by precision and recall. Precision indicates how many of the identified TLS instances are true tertiary lymphoid structures, and recall indicates how many of the true tertiary lymphoid structures the model detected. The digital pathology image processing system 110 may choose the desired precision or recall (with a corresponding trade-off between them) with which to tune the TLS detection model. Accordingly, the digital pathology image processing system 110 may tune/update the machine-learning model based on the selected precision or recall to bias the detection sensitivity and specificity of the model either towards detecting all true tertiary lymphoid structures (and possibly also detecting structures that are not true tertiary lymphoid structures), or towards detecting only true tertiary lymphoid structures (at the possible cost of missing a few true tertiary lymphoid structures). As an example and not by way of limitation, the digital pathology image processing system 110 may leverage a balance of tuning the TLS detection model against past model prediction results and tuning the TLS detection model according to the pathologists' annotations. As another example and not by way of limitation, the digital pathology image processing system 110 may further tune the TLS detection model (or a separate treatment outcome prediction model) based on a patient cohort dataset comprising patient clinical data for one or more types of cancer, annotated patient tissue sample images, patient treatment response data for one or more types of treatment, or any combination thereof.

In particular embodiments, when the digital pathology image of the tissue sample depicts one or more structures, identifying the one or more structures depicted within the digital pathology image of the tissue sample as being tertiary lymphoid structures may comprise the following steps. The digital pathology image processing system 110 may calculate, for each of the one or more structures detected and identified by the machine-learning model, a confidence score indicating a probability of the structure being a tertiary lymphoid structure. In some instances, the confidence scores for the one or more structures may be calculated based on, e.g., a precision and a recall for the TLS detection model by testing model predictions against ground truth in a training set of annotated slide images. The digital pathology image processing system 110 may then further determine, based on the one or more confidence scores, that one or more of the structures are actual tertiary lymphoid structures. In some instances, determining that the one or more structures are actual tertiary lymphoid structures may comprise, for example, comparing the confidence score for each structure to a predetermined confidence threshold. In some instances, the confidence score calculation and/or the predetermined confidence threshold may be tuned to adjust or maximize the precision and recall of the TLS detection model for a curated training set of annotated slide images. Thus, in some instances the confidence score may be a tuned confidence score, where the tuning accounts for a desired precision and recall in a curated set of annotated training slides.

In particular embodiments, the output of the TLS detection model may comprise confidence scores indicating the likelihood of the corresponding structures being tertiary lymphoid structures. In some instance, the digital pathology image processing system 110 may determine a threshold for comparison to the output of the TLS detection model to effectively identify tertiary lymphoid structures. For example, if the confidence score is larger than the threshold, the corresponding slide image may comprise a tertiary lymphoid structure. In particular embodiments, the digital pathology image processing system 110 may integrate the thresholding into a pathologist workflow by employing a more liberal threshold cutout. As an example and not by way of limitation, if the TLS detection model outputs a low confidence score for a given slide image, the digital pathology image processing system 110 may crop out image windows centered around the possible tertiary lymphoid structure, e.g., at high and low magnification. The digital pathology image processing system 110 may then pull all of those cropped slide images into a survey, which may be presented to a pathologist via a survey viewer displaying a slide image and a corresponding binary response field (i.e., is this a tertiary lymphoid structure or not?). With the aforementioned design, the workflow for pathologists may be transformed from having to open a slide image, review the image, find tertiary lymphoid structures, and draw circles around the identified TLS (or otherwise annotating them) to simply viewing a TLS prediction results survey, going through the survey results relatively rapidly, and providing a response in the binary response field. Thus, in some instances, the digital pathology image processing system 110 may use a fusion of TLS detection model predictions and a pathologist review to more accurately identify tertiary lymphoid structures.

In particular embodiments, the digital pathology image processing system 110 may tune the TLS detection model based on the confidence scores. A confidence score may be based on, for example, the intersection between a detected instance of a TLS and the corresponding annotation in a training image or pathologist-annotated image. As an example and not by way of limitation, if the detected TLS instance overlaps with the annotation, it may be considered a positive intersection. As can be seen, the intersection may indicate whether or not the TLS detection model prediction is correct. In particular embodiments, the digital pathology image processing system 110 may use intersection over union (IOU), a measure of the degree of overlap between a detected TLS instance and an annotation which may range from 0 to 1, to quantitatively evaluate the intersection. In some instances, the intersection over union may be calculated as the area of overlap, divided by the total are or union of the detected TLS instance/annotation pair. If the detected TLS instance and corresponding annotation are perfectly overlapping, the IOU may have a value of one. Correspondingly, the confidence score may be based on an intersection over union (IOU) calculation.

In particular embodiments, clusters of TLS instances that have high IOU values may tend to have annotations as tertiary lymphoid structures. The digital pathology image processing may determine how these TLS instances group together based on, e.g., their structure/numeric representations. In particular embodiments, the digital pathology image processing system 110 may generate, for each of the one or more structures detected by the machine-learning model, a numeric representation. The digital pathology image processing system 110 may then generate (using any of a variety of clustering algorithms such as k-means clustering method, hierarchical clustering, a mixture model, or any combination thereof) one or more clusters of structures based on the numeric representation associated with each of the one or more structures. As an example and not by way of limitation, if a TLS instance that shares a similar representation with a plurality of other TLS instances that have positive IOU has an IOU of zero, it may indicate that the pathologist may have missed it and may not have annotated it. As a result, the TLS detection model may additionally help identify TLS annotations missed by pathologists. The digital pathology image processing system 110 may further update the machine-learning model based on the one or more TLS clusters and the IOU values associated with each of the one or more clusters.

In particular embodiments, the digital pathology image processing system 110 may use continuous learning to improve the TLS detection model on a periodic or continuous basis. The digital pathology image processing system 110 may update and/or refine the training data set of tertiary lymphoid structures continuously, e.g., based on previous results. As an example and not by way of limitation, the digital pathology image processing system 110 may refine the training set by assigning more refined multi-class labels to tertiary lymphoid structures, e.g., immature tertiary lymphoid structure, mature tertiary lymphoid structure, one-point aggregate, etc. The TLS detection model trained on data comprising these refined labels may be able to perform more refined classification of TLS while segmenting the tertiary lymphoid structures from the slide images. By using more refined labels for multiple classes, the digital pathology image processing system 110 may also generate more robust numerical representations for the tertiary lymphoid structures and enable the machine learning model to more a clearly distinguish between immature tertiary lymphoid structures, mature tertiary lymphoid structures, and artifacts, etc.

The digital pathology image processing system 110 may also scale up the training data set by collecting more data. As an example and not by way of limitation, suppose the TLS detection model is trained solely based on images of mature tertiary lymphoid structures. The model may then still exhibit uncertainty about whether a slide image comprises an immature tertiary lymphoid structure, i.e., likely identifying an immature tertiary lymphoid structure as a mature tertiary lymphoid structure. For slide images of chronic disease, the TLS detection model may be biased towards detecting structures that look like a tertiary lymphoid structure. The digital pathology image processing system 110 may leverage that bias to collect additional training data. The digital pathology image processing system 110 may then prescreen whole slide images comprising these biased structures, crop them, and collect descriptions of these structures from pathologists. For example, the descriptions provided by the pathologists may comprise mature tertiary lymphoid structures, immature tertiary lymphoid structures, artifacts, none of the above, etc., thereby creating new target labels not previously collected for retraining of the TLS detection model.

### Machine Learning Methods

In some instances of the disclosed methods, the method may comprise the use of one or more machine learning methods and/or statistical analysis methods to perform pre-processing of images (e.g., image segmentation to identify objects of interest and extract image feature data) in addition to subsequently performing TLS detection and identification. Any of a variety of machine learning models may be used in implementing the disclosed methods. For example, the machine learning models(s) employed may comprise a supervised learning model, an unsupervised learning model, a semi-supervised learning model, a deep learning model, etc., or any combination thereof.

### Supervised Learning Models

In the context of the present disclosure, supervised learning models are models that rely on the use of a set of labeled training data to infer the relationship between a set of input data (e.g., image data for identified candidate structures) and a classification of the input data into to a specified set of user-specified classes (e.g., tertiary lymphoid structures). The training data used to “teach” the supervised learning model comprises a set of paired training examples, e.g., where each example comprises an annotated slide image that depicts a tertiary lymphoid structure. Examples of supervised learning models include support vector machines (SVMs), artificial neural networks (ANNs), etc.

### Unsupervised Learning Models

In the context of the present disclosure, unsupervised learning models are models used to draw inferences from training datasets consisting of image feature datasets that are not paired with labeled tissue phenotype classification data. One example of a commonly used unsupervised learning models is cluster analysis, which is often used for exploratory data analysis to find hidden patterns or groupings in multi-dimensional data sets. Other examples of unsupervised learning models include, but are not limited to, artificial neural networks, association rule learning models, etc.

### Semi-Supervised Learning Models:

In the context of the present disclosure, semi-supervised learning models are models that make use of both labeled and unlabeled image patch data for training (typically using a relatively small amount of labeled data with a larger amount of unlabeled data).

### Artificial Neural Networks and Deep Learning Models:

In the context of the present disclosure, artificial neural networks (ANNs) are models which are inspired by the structure and function of the human brain. Artificial neural networks comprise an interconnected group of nodes organized into multiple layers. For example, the ANN architecture may comprise at least an input layer, one or more hidden layers, and an output layer. Deep learning models are large artificial neural networks comprising many hidden layers of coupled “nodes” between the input layer and output layer that may be used, for example, to map image patch data or image feature data to tissue phenotype classification decisions.

The ANN may comprise any total number of layers, and any number of hidden layers, where the hidden layers function as trainable feature extractors that allow mapping of a set of input data to a preferred output value or set of output values. Each layer of the neural network comprises a number of nodes (or “neurons”). A node receives input that comes either directly from the input data (e.g., image patch data or image feature data derived from image patch data) or from the output of nodes in previous layers, and performs a specific operation, e.g., a summation operation.

In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a neuron may be gated using a threshold or activation function, ƒ, which may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, can be “taught” or “learned” in a training phase using one or more sets of training data. For example, the parameters may be trained using the input data from a training data set and a gradient descent or backward propagation method so that the output value(s) (e.g., a TLS classification decision) that the ANN computes are consistent with the examples included in the training data set. The adjustable parameters of the model may be obtained using, e.g., a back propagation neural network training process that may or may not be performed using the same hardware as that used for processing images and/or performing tissue sample.

Other specific types of deep machine learning models, e.g., convolutional neural networks (CNNs) (often used for the processing of image data from machine vision systems) may also be used in implementing the disclosed methods and systems. CNN are commonly composed of layers of different types: convolution, pooling, upscaling, and fully-connected node layers. In some cases, an activation function such as rectified linear unit may be used in some of the layers. In a CNN architecture, there can be one or more layers for each type of operation performed. A CNN architecture may comprise any number of layers in total, and any number of layers for the different types of operations performed. The simplest convolutional neural network architecture starts with an input layer followed by a sequence of convolutional layers and pooling layers, and ends with fully-connected layers. Each convolution layer may comprise a plurality of parameters used for performing the convolution operations. Each convolution layer may also comprise one or more filters, which in turn may comprise one or more weighting factors or other adjustable parameters. In some instances, the parameters may include biases (i.e., parameters that permit the activation function to be shifted). In some cases, the convolutional layers are followed by a layer of ReLU activation function. Other activation functions can also be used, for example the saturating hyperbolic tangent, identity, binary step, logistic, arcTan, softsign, parameteric rectified linear unit, exponential linear unit, softPlus, bent identity, softExponential, Sinusoid, Sine, Gaussian, the sigmoid function and various others. The convolutional, pooling and ReLU layers may function as learnable features extractors, while the fully connected layers may function as a machine learning classifier. As with other artificial neural networks, the convolutional layers and fully-connected layers of CNN architectures typically include various adjustable computational parameters, e.g., weights, bias values, and threshold values, that are trained in a training phase as described above.

### Autoencoders

In some instances, implementation of the disclosed methods and systems may comprise the use of an autoencoder model. Autoencoders (also sometimes referred to as an auto-associator or Diabolo networks) are artificial neural networks used for unsupervised, efficient mapping of input data, e.g., image feature data, to an output value, e.g., a numeric representation of the image feature data. Autoencoders are often used for the purpose of dimensionality reduction, i.e., the process of reducing the number of random variables under consideration by deducing a set of principal component variables. Dimensionality reduction may be performed, for example, for the purpose of feature selection (e.g., selection of the most relevant subset of the image features presented in the original image feature data set) or feature extraction (e.g., transformation of image feature data in the original, multi-dimensional image space to a space of fewer dimensions as defined, e.g., by a series of feature parameters, Zr).

Any of a variety of different autoencoder models known to those of skill in the art may be used in the disclosed methods and systems. Examples include, but are not limited to, stacked autoencoders, de-noising autoencoders, variational autoencoders, or any combination thereof. Stacked autoencoders are neural networks consisting of multiple layers of sparse autoencoders in which the output of each layer is wired to the input of the successive layer. Variational autoencoders (VAEs) are autoencoder models that use the basic autoencoder architecture, but that make strong assumptions regarding the distribution of latent variables. They use a variational approach for latent representation learning, which results in an additional loss component, and may require the use of a specific training method called Stochastic Gradient Variational Bayes (SGVB).

### Clustering Methods

In some instances, the disclosed methods and systems may comprise the use of a clustering method to, for example, cluster detected tertiary lymphoid structures based on their numeric representations. Any of a variety of clustering methods known to those of skill in the art may be used. Examples of suitable clustering methods include, but are not limited to, k-means clustering methods, hierarchical clustering methods, mean-shift clustering methods, density-based spatial clustering methods, expectation-maximization clustering methods, and mixture model (e.g., mixtures of Gaussians) clustering methods.

K-means clustering methods are unsupervised machine learning methods used to partition n data points into k non-overlapping clusters such that each data point belongs to only one cluster and data points in the same cluster are characterized by, e.g., similar features or numeric representations, while data points in different clusters are characterized by very different features. Data points are assigned to a cluster such that the sum of the squared distances between the data points belonging to the cluster and the cluster's centroid (or arithmetic mean of all the data points that belong to that cluster) is minimized.

Hierarchical clustering methods are methods that also group data points into groups or clusters. The objective is to identify a set of clusters that characterize the original data set, where each cluster is distinct from each other cluster the data points within each cluster share broadly similar features, and each data point belongs to a single cluster. Initially, each data point is treated as a separate cluster. A distance matrix for pairs of data points is calculated, and the method then repeats the steps of: (i) identifying the two clusters that are closest together, and (ii) merging the two most similar clusters. The iterative process continues until all similar clusters have been merged.

Gaussian mixture models are probabilistic models that assume all data points in a data set may be represented by a mixture of a finite number of Gaussian distributions with unknown peak height, position, or standard deviations. The approach is similar to generalizing a k-means clustering method to incorporate information about the covariance structure of the data as well as the centers of the latent Gaussians.

### Machine Learning Training Data

The type of training data used for training a machine learning model for use in the disclosed methods and systems will depend on, for example, whether a supervised or unsupervised approach is taken as well as on the objective to be achieved. In some instances, one or more training data sets may be used to train the model(s) in a training phase that is distinct from that of the application (or deployment) phase. In some instances, training data may be continuously updated and used to update the machine learning model(s) in a local or distributed network of one or more deployed pathology image analysis systems in real time. In some cases, the training data may be stored in a training database that resides on a local computer or server. In some cases, the training data may be stored in a training database that resides online or in the cloud.

In some instances, e.g., classification of candidate structures apparent in a slide image into tertiary lymphoid structures of different maturation state, the training data may comprise data derived from a series of one or more pre-processed, segmented whole slide images, or portions thereof, where each image of the series comprises an image of an individual tissue sample. In some instances, a machine learning model may be used to perform all or a portion of the pre-processing and segmentation of the series of one or more tissue sample images as well as the subsequent analysis (e.g., TLS detection and prediction of patient outcomes). In some cases, the training data set may include other types of input data, e.g., phenotype, genotype, and/or nucleic acid sequencing data for the tissue sample, patient clinical data, etc.

### Computer Systems

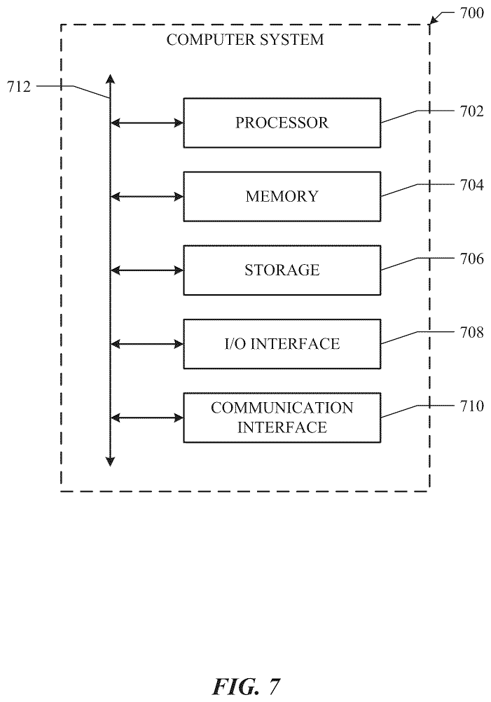

FIG. 7 illustrates an example computer system 700. In particular embodiments, one or more computer systems 700 perform one or more steps of one or more methods described or illustrated herein. In particular embodiments, one or more computer systems 700 provide functionality described or illustrated herein. In particular embodiments, software running on one or more computer systems 700 performs one or more steps of one or more methods described or illustrated herein or provides functionality described or illustrated herein. Particular embodiments include one or more portions of one or more computer systems 700. Herein, reference to a computer system may encompass a computing device, and vice versa, where appropriate. Moreover, reference to a computer system may encompass one or more computer systems, where appropriate.

This disclosure contemplates any suitable number of computer systems 700. This disclosure contemplates computer system 700 taking any suitable physical form. As example and not by way of limitation, computer system 700 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 700 may include one or more computer systems 700; be unitary or distributed; span multiple locations; span multiple machines; span multiple data centers; or reside in a cloud, which may include one or more cloud components in one or more networks. Where appropriate, one or more computer systems 700 may perform without substantial spatial or temporal limitation one or more steps of one or more methods described or illustrated herein. As an example and not by way of limitation, one or more computer systems 700 may perform in real time or in batch mode one or more steps of one or more methods described or illustrated herein. One or more computer systems 700 may perform at different times or at different locations one or more steps of one or more methods described or illustrated herein, where appropriate.

In particular embodiments, computer system 700 includes a processor 702, memory 704, storage 706, an input/output (I/O) interface 708, a communication interface 710, and a bus 712. Although this disclosure describes and illustrates a particular computer system having a particular number of particular components in a particular arrangement, this disclosure contemplates any suitable computer system having any suitable number of any suitable components in any suitable arrangement.

In particular embodiments, processor 702 includes hardware for executing instructions, such as those making up a computer program. As an example and not by way of limitation, to execute instructions, processor 702 may retrieve (or fetch) the instructions from an internal register, an internal cache, memory 704, or storage 706; decode and execute them; and then write one or more results to an internal register, an internal cache, memory 704, or storage 706. In particular embodiments, processor 702 may include one or more internal caches for data, instructions, or addresses. This disclosure contemplates processor 702 including any suitable number of any suitable internal caches, where appropriate. As an example and not by way of limitation, processor 702 may include one or more instruction caches, one or more data caches, and one or more translation lookaside buffers (TLBs). Instructions in the instruction caches may be copies of instructions in memory 704 or storage 706, and the instruction caches may speed up retrieval of those instructions by processor 702. Data in the data caches may be copies of data in memory 704 or storage 706 for instructions executing at processor 702 to operate on; the results of previous instructions executed at processor 702 for access by subsequent instructions executing at processor 702 or for writing to memory 704 or storage 706; or other suitable data. The data caches may speed up read or write operations by processor 702. The TLBs may speed up virtual-address translation for processor 702. In particular embodiments, processor 702 may include one or more internal registers for data, instructions, or addresses. This disclosure contemplates processor 702 including any suitable number of any suitable internal registers, where appropriate. Where appropriate, processor 702 may include one or more arithmetic logic units (ALUs); be a multi-core processor; or include one or more processors 702. Although this disclosure describes and illustrates a particular processor, this disclosure contemplates any suitable processor.

In particular embodiments, memory 704 includes main memory for storing instructions for processor 702 to execute or data for processor 702 to operate on. As an example and not by way of limitation, computer system 700 may load instructions from storage 706 or another source (such as, for example, another computer system 700) to memory 704. Processor 702 may then load the instructions from memory 704 to an internal register or internal cache. To execute the instructions, processor 702 may retrieve the instructions from the internal register or internal cache and decode them. During or after execution of the instructions, processor 702 may write one or more results (which may be intermediate or final results) to the internal register or internal cache. Processor 702 may then write one or more of those results to memory 704. In particular embodiments, processor 702 executes only instructions in one or more internal registers or internal caches or in memory 704 (as opposed to storage 706 or elsewhere) and operates only on data in one or more internal registers or internal caches or in memory 704 (as opposed to storage 706 or elsewhere). One or more memory buses (which may each include an address bus and a data bus) may couple processor 702 to memory 704. Bus 712 may include one or more memory buses, as described below. In particular embodiments, one or more memory management units (MMUs) reside between processor 702 and memory 704 and facilitate accesses to memory 704 requested by processor 702. In particular embodiments, memory 704 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 704 may include one or more memories 704, where appropriate. Although this disclosure describes and illustrates particular memory, this disclosure contemplates any suitable memory.

In particular embodiments, storage 706 includes mass storage for data or instructions. As an example and not by way of limitation, storage 706 may include a hard disk drive (HDD), a floppy disk drive, flash memory, an optical disc, a magneto-optical disc, magnetic tape, or a Universal Serial Bus (USB) drive or a combination of two or more of these. Storage 706 may include removable or non-removable (or fixed) media, where appropriate. Storage 706 may be internal or external to computer system 700, where appropriate. In particular embodiments, storage 706 is non-volatile, solid-state memory. In particular embodiments, storage 706 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 706 taking any suitable physical form. Storage 706 may include one or more storage control units facilitating communication between processor 702 and storage 706, where appropriate. Where appropriate, storage 706 may include one or more storages 706. Although this disclosure describes and illustrates particular storage, this disclosure contemplates any suitable storage.

In particular embodiments, I/O interface 708 includes hardware, software, or both, providing one or more interfaces for communication between computer system 700 and one or more I/O devices. Computer system 700 may include one or more of these I/O devices, where appropriate. One or more of these I/O devices may enable communication between a person and computer system 700. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 708 for them. Where appropriate, I/O interface 708 may include one or more device or software drivers enabling processor 702 to drive one or more of these I/O devices. I/O interface 708 may include one or more I/O interfaces 708, where appropriate. Although this disclosure describes and illustrates a particular I/O interface, this disclosure contemplates any suitable I/O interface.

In particular embodiments, communication interface 710 includes hardware, software, or both providing one or more interfaces for communication (such as, for example, packet-based communication) between computer system 700 and one or more other computer systems 700 or one or more networks. As an example and not by way of limitation, communication interface 710 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 710 for it. As an example and not by way of limitation, computer system 700 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 700 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 700 may include any suitable communication interface 710 for any of these networks, where appropriate. Communication interface 710 may include one or more communication interfaces 710, where appropriate. Although this disclosure describes and illustrates a particular communication interface, this disclosure contemplates any suitable communication interface.

In particular embodiments, bus 712 includes hardware, software, or both coupling components of computer system 700 to each other. As an example and not by way of limitation, bus 712 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 712 may include one or more buses 712, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

What is claimed is:

1. A method comprising, by a digital pathology image processing system:
accessing a digital pathology image that depicts a tissue sample from a subject under a treatment;
detecting, based on a machine-learning model, one or more tertiary lymphoid structures depicted within the digital pathology image of the tissue sample;
determining, for the one or more detected tertiary lymphoid structures, descriptive information associated with the detected tertiary lymphoid structures, wherein the descriptive information comprises at least a maturation state associated with each of the detected tertiary lymphoid structures; and
determining, based on the detected tertiary lymphoid structures and the descriptive information, an outcome of the subject in response to the treatment.

2. The method of claim 1, further comprising:
identifying, for each of the one or more tertiary lymphoid structures, an area comprising the tertiary lymphoid structure; and
providing instructions for outlining the area comprising the tertiary lymphoid structure in the digital pathology image, wherein the outlining comprises a polygonal shape.

3. The method of claim 1, wherein the digital pathology image of the tissue sample depicts one or more structures, and wherein the method further comprises:
generating, for each of the one or more structures by the machine-learning model, a numeric representation, wherein detecting the one or more tertiary lymphoid structures comprises determining one or more of the structures as the one or more tertiary lymphoid structures based on the respective numeric representations associated with the one or more of the structures.

4. The method of claim 1, wherein each of the one or more detected tertiary lymphoid structures is associated with a numeric representation generated by the machine-learning model, and wherein the method further comprises:
determining the maturation state associated with each of the detected tertiary lymphoid structures based on their respective numeric representations.

5. The method of claim 1, wherein the digital pathology image of the tissue sample depicts one or more structures, and wherein detecting the one or more tertiary lymphoid structures depicted within the digital pathology image of the tissue sample comprises:
determining whether each of the one or more structures comprises a germinal center; and
based on the determining of whether each of the one or more structures comprises a germinal center:
if at least one of the one or more structures comprises a germinal center, determining the at least one structure as a tertiary lymphoid structure;
else:
analyzing an interaction between a presence of each of the one or more structures and one or more features associated with the structure; and
determining whether each of the one or more structures is a tertiary lymphoid structure based on the analyzed interaction.

6. The method of claim 1, wherein the digital pathology image of the tissue sample depicts one or more structures, and wherein detecting the one or more tertiary lymphoid structures depicted within the digital pathology image of the tissue sample comprises:
calculating, for each of the one or more structures identified by the machine-learning model, a confidence score based on a precision and recall for the machine-learning model, wherein the confidence score indicates a probability of the structure being a tertiary lymphoid structure; and
determining, based on the one or more confidence scores, that one or more of the structures are tertiary lymphoid structures.

7. The method of claim 6, wherein the calculation used to determine confidence scores has been tuned to maximize the precision and recall of the machine-learning model when applied to a training set of annotated slide images.

8. The method of claim 6, wherein the method further comprises:
generating, for each of the one or more structures by the machine-learning model, a numeric representation;
generating, one or more clusters of structures based on the numeric representation associated with each of the one or more structures; and
updating the machine-learning model based on the one or more clusters associated with each of the one or more structures.

9. The method of claim 1, wherein each of the determined one or more tertiary lymphoid structures is associated with a numeric representation, wherein the method further comprises:
generating, one or more clusters of the detected tertiary lymphoid structures based on the numeric representation associated with each of the detected tertiary lymphoid structures;
wherein determining the outcome of the subject in response to the treatment is further based on the one or more clusters.

10. The method of claim 1, wherein the tissue sample is associated with one or more tumors, wherein the method further comprises:
determining a type of the tissue sample;
generating a tissue image mask for the tissue sample;
identifying at least one tumor region within the tissue image mask; and
determining a type of the at least one tumor region within the tissue image mask;
wherein determining the outcome of the subject in response to the treatment is further based on the type of the tissue sample, the tissue image mask, and the type of the at least one tumor within the tissue image mask.

11. The method of claim 1, wherein the tissue sample is associated with one or more tumors, and wherein the descriptive information further comprises one or more of:
a number of the detected tertiary lymphoid structures;
a number of detected tertiary lymphoid structures that are associated with image markers;
a number of detected tertiary lymphoid structures outside a tumor region;
a ratio between a number of detected tertiary lymphoid structures located inside the tumor region and a number of detected tertiary lymphoid structures located outside the tumor region;
an average distance of the detected tertiary lymphoid structures to the tumor region or a boundary thereof;
a size of each of the detected tertiary lymphoid structures;
a percentage of a total tissue sample area that comprises the detected tertiary lymphoid structures; or
an average distance between any given pair of detected tertiary lymphoid structures.

12. The method of claim 1, wherein the machine-learning model is trained based on a plurality of training data, wherein each training data point comprises a slide image of a tissue sample and a corresponding annotation of tertiary lymphoid structures identified within that tissue sample.

13. The method of claim 12, further comprising training the machine-learning model, wherein the training comprises:
applying one or more data augmentations to each slide image of a training data set, wherein the one or more data augmentations are based on one or more of brightness, hue, saturation, cropping, clipping, flipping, rotation, or a mean pixel density in a color channel.

14. The method of claim 12, wherein the machine-learning model is based on one or more neural networks, and wherein the method further comprises training the machine-learning model, and wherein the training comprises:
generating, by the one or more neural networks, one or more initial bounding boxes for each slide image, wherein each initial bounding box is associated with an initial aspect ratio and an initial size;
adjusting, based on characteristics of tertiary lymphoid structures, the initial aspect ratio and the initial size associated with each of the initial bounding box; and
training the machine-learning model based on slide image data using the adjusted bounding boxes.

15. The method of claim 12, further comprising training the machine-learning model, wherein the training comprises:
identifying at least two tertiary lymphoid structures within at least one slide image of the training data;
generating a cropped image from the at least one slide image by cropping the at least one slide image to make the at least two tertiary lymphoid structures centered; and
training the machine-learning model based in part on the cropped image.

16. One or more computer-readable non-transitory storage media embodying software that is operable when executed to:
access a digital pathology image that depicts a tissue sample from a subject under a treatment;
detect, based on a machine-learning model, one or more tertiary lymphoid structures depicted within the digital pathology image of the tissue sample;
determine, for the one or more detected tertiary lymphoid structures, descriptive information associated with the detected tertiary lymphoid structures, wherein the descriptive information comprises at least a maturation state associated with each of the detected tertiary lymphoid structures; and
determine, based on the detected tertiary lymphoid structures and the descriptive information, an outcome of the subject in response to the treatment.

17. The media of claim 16, wherein the software is further operable when executed to:
identify, for each of the one or more tertiary lymphoid structures, an area comprising the tertiary lymphoid structure; and
provide instructions for outlining the area comprising the tertiary lymphoid structure in the digital pathology image, wherein the outlining comprises a polygonal shape.

18. The media of claim 16, wherein the tissue sample comprises one or more structures, wherein the software is further operable when executed to:
generate, for each of the one or more structures by the machine-learning model, a numeric representation, wherein detecting the one or more tertiary lymphoid structures comprises determining one or more of the structures as the one or more tertiary lymphoid structures based on the respective numeric representations associated with the one or more of the structures.

19. A system comprising: one or more processors; and a non-transitory memory coupled to the processors comprising instructions executable by the processors, the processors operable when executing the instructions to:
access a digital pathology image that depicts a tissue sample from a subject under a treatment;
detect, based on a machine-learning model, one or more tertiary lymphoid structures depicted within the digital pathology image of the tissue sample;
determine, for the one or more detected tertiary lymphoid structures, descriptive information associated with the detected tertiary lymphoid structures, wherein the descriptive information comprises at least a maturation state associated with each of the detected tertiary lymphoid structures; and
determine, based on the detected tertiary lymphoid structures and the descriptive information, an outcome of the subject in response to the treatment.

20. The system of claim 19, wherein the processors are further operable when executing the instructions to:
identify, for each of the one or more tertiary lymphoid structures, an area comprising the tertiary lymphoid structure; and
provide instructions for outlining the area comprising the tertiary lymphoid structure in the digital pathology image, wherein the outlining comprises a polygonal shape.
